SEQUENCE LISTING (4 sequences)

SEQ ID NO: 1 - DNA, 25 bp, synthetic construct
  misc_feature 1..25: note = GP6-16 Primer
  source 1..25: mol_type = other DNA
gaaaaataaactgtaaatcatattc

SEQ ID NO: 2 - DNA, 50 bp, synthetic construct
  misc_feature 1..50: note = GP6-50 primer
  source 1..50: mol_type = other DNA
gcagttaaggttattttgcacagttgaaaaataaactgtaaatcatattc

SEQ ID NO: 3 - DNA, 23 bp, synthetic construct
  misc_feature 1..23: note = Primer for amplifying HPV
  source 1..23: mol_type = other DNA
tttntnacnkkngtngayacnac

SEQ ID NO: 4 - DNA, 28 bp, synthetic construct
  misc_feature 1..28: note = Primer for amplifying HPV
  source 1..28: mol_type = other DNA
gaaanaynaantgyannwcrwaytcytc

(19) United States
(12) Patent Application Publication
(10) Pub. No.: US 2023/0272492 A1
(43) Pub. Date: Aug. 31, 2023

(54) BEAD COMPLEX FOR DETECTION OF NUCLEIC ACID MOLECULES IN BIOLOGICAL SAMPLES AND METHOD OF DETECTING NUCLEIC ACID USING THE SAME

(71) Applicant: SML GENETREE CO., LTD., Seoul (KR)

(72) Inventors: Yo Han JEONG, Seoul (KR); Jeong Ju LEE, Seoul (KR); So Hyeon PARK, Seoul (KR); Kyung Ah HWANG, Seoul (KR); Ji Hoon AHN, Seoul (KR)

(73) Assignee: SML GENETREE CO., LTD., Seoul (KR)

(21) Appl. No.: 17/985,414

(22) Filed: Nov. 11, 2022

Related U.S. Application Data
(63) Continuation of application No. PCT/KR2022/011953, filed on Aug. 10, 2022.

(30) Foreign Application Priority Data
Aug. 10, 2021 (KR) 10-2021-0105130
Apr. 7, 2022 (KR) 10-2022-0043477
Apr. 15, 2022 (KR) 10-2022-0046809

(51) Int. Cl. C12Q 1/70; C12Q 1/6855

(52) U.S. Cl. CPC C12Q 1/708; C12Q 1/6855

(57) ABSTRACT

Provided is a bead complex for detecting a nucleic acid molecule in a biological sample, and a method of detecting nucleic acid using the same. According to an aspect, the bead complex may effectively isolate, extract, and detect a target nucleic acid molecule in a biological sample, and by analyzing whether a target nucleic acid molecule is present in a biological sample, there is an effect of improving sensitivity of the assay.

### CROSS-REFERENCE TO RELATED APPLICATIONS

This application is a continuation of International Application No. PCT/KR2022/011953 filed on Aug. 10, 2022, which claims priority under 35 U.S.C. § 119 to Korean Patent Application No. 10-2021-0105130 filed on Aug. 10, 2021, Korean Patent Application No. 10-2022-0043477 filed on Apr. 7, 2022, and Korean Patent Application No. 10-2022-0046809 filed on Apr. 15, 2022, in the Korean Intellectual Property Office, the disclosure of which is incorporated by reference herein in its entirety.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created Aug. 4, 2022, is named “PX220228PCT.xml” and is 6 Kb in size.

### TECHNICAL FIELD

The present disclosure relates to a bead complex for detection of a nucleic acid molecule in a biological sample and a method of detecting nucleic acid using the same.

### BACKGROUND ART

Methods of extracting nucleic acids from a sample are largely divided into column methods and methods using magnetic beads. Purification using a column is performed in a way that cells present in a sample are lysed, collected using a column, and then tested through polymerase chain reaction (PCR). Purification using magnetic beads is performed by collecting and purifying desired nucleic acids using surface charges of magnetic beads having charge-to-charge interaction with the nucleic acids. However, since these methods non-specifically collect various proteins, inhibitors, genomic DNA, and RNA in a sample, additional purification processes are required. In addition, accuracy of PCR reaction is low due to various inhibitors.

For example, the most currently performed examination method for the diagnosis of human papillomavirus (HPV), a major pathogen that causes cervical cancer, is a cytodiagnosis examination in which a cotton swab is inserted into the cervix to collect cells and then the cells are stained to observe presence or absence of cancer cells. However, in the case of cytodiagnosis, sensitivity is rather low at 50% to 70%, and to compensate for this, additional HPV genetic tests are performed to determine presence or absence of HPV virus in cervical cells. When a HPV test result is positive, even when a cytodiagnosis result is negative, probability of cervical cancer is high, and therefore, additional PCR tests are currently widely used.

Currently, there are two methods mainly used for HPV screening: a Pap smear test and a pad-type detection method. A Pap test is a method of identifying presence or absence of HPV virus through PCR after collecting cells by inserting a cotton swab into the cervix. A pad-type detection is a method of identifying presence or absence of HPV virus by PCR by collecting HPV virus in secretions using a pad worn by a subject.

The pap test has disadvantages of being invasive and requiring specialized medical staff for detection. The pad-type detection method has disadvantages in that it is impossible for the subject to take a shower or wash the vagina 24 hours to 72 hours before the test and the subject has to wear a pad for a long time. Therefore, if it is possible to detect presence of HPV by detecting DNA or RNA present in urine, the disadvantages of the existing test methods may be sufficiently compensated and HPV infection may be identified within a relatively short time. However, various inhibitors, proteins, DNA, and RNA are present in urine in very small amounts, compared to the amount of urine. The purification method using a column and the purification method using magnetic beads, which are currently in use, are methods that purify all substances with a charge in urine. Therefore, since impurities other than the intended nucleic acid are separated together, accuracy of PCR reaction is deteriorated, an additional purification process is required, and there is a problem of lowered specificity of PCR reaction.

### SUMMARY

### Technical Problem

An aspect is to provide a bead complex capable of effectively detecting nucleic acid molecules present in a biological sample by specifically collecting and purifying the same.

Another aspect is to provide a method of preparing the bead complex capable of effectively detecting nucleic acid molecules present in a biological sample by specifically collecting and purifying the same.

Still another aspect is to provide a method of detecting target nucleic acid molecules in a biological sample including urine using the bead complex.

### Technical Solution to Problem

An aspect provides a bead complex in which one end of an oligo-nucleotide, which binds specifically to a target nucleic acid molecule, is conjugated to a surface of a bead, and the surface of the bead and the oligo-nucleotide may be bonded by a structure of Formula 1 below:

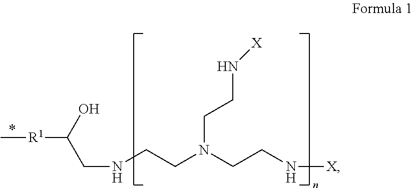

wherein, X in the formula is hydrogen or

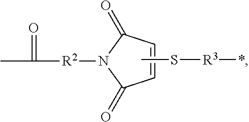

and at least one X is

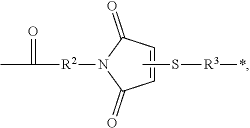

R¹ in the formula is a direct bond or a C₁-C₂₀ aliphatic hydrocarbon group,
R² and R³ in the formula are each independently a C₂-C₂₀ aliphatic hydrocarbon group,
n in the formula is an integer of 1 or more, for example, an integer of 1 to 100,000, 1 to 10,000, 1 to 1,000, 1 to 100, 1 to 50, 1 to 40, 1 to 30, 1 to 20, or 1 to 10, and
the asterisk on the left of R¹ indicates a site connected to the bead surface, and the asterisk on the right of R³ indicates a site connected to the end of the oligo-nucleotide.

R¹ in Formula 1 may be a direct bond or an aliphatic hydrocarbon group selected from the group consisting of a C₁-C₂₀ alkyl group, a C₂-C₂₀ alkenyl group, a C₂-C₂₀ alkynyl group, and a C₁-C₂₀ alkyl ether group. In an embodiment, the aliphatic hydrocarbon group constituting R¹ of Formula 1 may be selected from the group consisting of a C₁-C₁₀ alkyl group, a C₂-C₂₀ alkenyl group, a C₂-C₂₀ alkynyl group, and a C₁-C₂₀ alkyl ether group, but is not limited thereto. In addition, in Formula 1, R² and R³ may each independently be a C₂-C₂₀ alkylene group, for example, a C₂-C₁₀ alkylene group, or a C₅-C₁₀ alkylene group, but is not limited thereto.

In an embodiment, R² may be a C₂-C₁₀ alkylene group.

In an aspect, disclosed is a bead complex, in which one end of an oligo-nucleotide binding specifically to a target nucleic acid molecule is conjugated to a surface of a nano-bead, and a linker having a structure of Formula 7 below is interposed between the surface of the nano-bead and the oligo-nucleotide:

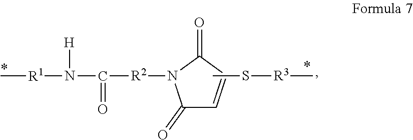

(in Formula 7, R¹ is a direct bond or a C₁-C₂₀ aliphatic hydrocarbon group; R² and R³ are each independently a C₃-C₂₀ divalent aliphatic hydrocarbon group linkers; the asterisk on the left of R¹ represents a site connected to the bead surface, and the asterisk on the right of R³ indicates a site connected to the end of the oligo-nucleotide.)

For example, R² and R³ in Formula 7 may each independently be a C₃-C₂₀ alkylene group.

The term “bead”, used herein, may be used interchangeably with “particle”.

In an embodiment, the bead may be made of an inorganic material.

The inorganic material may be at least one non-metallic material selected from the group consisting of iron oxide, silica, glass, and combinations thereof, and a metal material selected from the group consisting of gold, silver, copper, combinations thereof, and alloys thereof.

The inorganic material may be selected from the group consisting of iron oxide, silica, gold, silver, copper, and combinations thereof.

In an embodiment, the bead may be made of an organic material.

The organic material may be a polymer resin selected from the group consisting of polystyrene, polypropylene, polyethylene, poly acrylamide, combinations thereof, and copolymers thereof, and at least one polysaccharide selected from the group consisting of fullulane, fullulane acetate, cellulose, hydroxypropylmethylcellulose, agarose, chitosan, combinations thereof, and copolymers thereof.

In an embodiment, the bead may be a magnetized bead.

The beads may have a diameter of 0.1 μm to 100 μm, preferably 0.2 μm to 10 μm, or more preferably 0.4 μm to 1 μm.

The magnetization value of the magnetized beads may be 0.1 emu/g to 1,000 emu/g, preferably, 1 emu/g to 100 emu/g, or more preferably, 5 emu/g to 10 emu/g.

In an embodiment, the bead is not limited in its shape, and any shape, for example, a spherical shape, a rod shape, a wire shape (linear), a flat shape, or an amorphous shape is possible.

In an embodiment, the bead may be one modified with an amino group, a thiol group, an aldehyde group, a carboxy group, a hydroxyl group, a maleimide group, or a C₂-C₁₀ alkenyl group.

In an embodiment, the conjugation may be through any one bond selected from an amide bond, a formamide bond, an ester bond, a thioester bond, a disulfide bond, an ether bond, and a glycosidic bond.

The target nucleic acid molecule may be derived from a pathogen, cancer, or tumor, and may be in the form of DNA or RNA.

The target nucleic acid molecule may be derived from a cancer cell or a pathogen.

For example, the pathogen may include, but is not limited to, pathogenic viruses and pathogenic bacteria.

For example, the target nucleic acid molecule may be derived from human papillomavirus.

The target nucleic acid molecule may be a nucleic acid molecule as a marker indicating the onset of cancer or the infection of a pathogen-related disease. In an example, the cancer or tumor includes, but is not limited to, stomach cancer, lung cancer, liver cancer, colorectal cancer, small intestine cancer, skin cancer, pancreatic cancer, and prostate cancer. In an optional aspect, the target nucleic acid molecule derived from cancer or tumor may be a nucleic acid molecule encoding a protein or a peptide that is a tumor antigen or a fragment thereof, a variant or a derivative thereof.

The nucleic acid molecule derived from a pathogen may be a nucleic acid molecule that is a factor or a marker indicating whether or not the pathogen is infected. For example, a nucleic acid molecule derived from a pathogen may include a nucleic acid molecule derived from a pathogenic virus, a pathogenic bacterium, or a pathogenic protozoan. In an example, the target nucleic acid molecule may be a nucleic acid molecule encoding a protein or a peptide including a pathogenic antigen or a fragment thereof, a variant or a derivative thereof.

The pathogenic antigen may be derived from a pathogenic organism, such as a bacterium, a virus or a protozoan, that elicits an immunological response in a subject, in particular, a mammalian subject, in particular, a human. In an example, the pathogenic antigen may be a surface antigen or a portion thereof located on the surface of a bacterium, a virus or a protozoic organism.

In an example, the pathogenic antigen may be a peptidic or proteinic antigen derived from a pathogen associated with an infectious disease. More specifically, the pathogenic antigen may be derived from Acinetobacter baumannii, Anaplasma genus, Anaplasma phagocytophilum, Ancylostoma braziliense, Ancylostoma duodenale, Armayobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdor Berry (Borrelia burgdorferi), Borrelia genus, Borrelia spp, Brucella genus, Brugia malayi, Bunyaviridae family, Buryaviridae family, Burkholderia cepacia, and other Burkholderia species, Burkholderia mallei, Burkholderia pseudomalei, Caliciviridae family, Campylobacter genus, maydida albimays, maydida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium spp, Clostridium tetani, Coccidioides spp, coronaviruses, Corynebacterium diphtheria, Coxiella burnetii, Crimean-Congo hemorrhagic fever virus, Cryptococcus neoformans, Cryptosporidium genus, Cytomegalovirus (CMV), dengue viruses (DEN-1, DEN-2, DEN-3 and DEN-4), Dientamoeba fragilis, ebolavirus (EBOV), Echinococcus genus, Ehrlicia chaffeensis, Ehrlichia ewingii, Ehrlichia genus, Entamoeba histolytica, Enterococcus genus, Enterovirus genus, enteroviruses, mainly coxsackie A virus and enterovirus 71 (EV71), Epidermophyton spp, Epstein-Barr Virus (EBV), Escherichia coli O157:H7, O111 and O104:H4, Fasciola hepatica and Fasciola gigantica, FFI prion, Filarioidea superfamily, Flaviviruses, Francisella tularensis, Fusobacterium genus, Geotrichum maydidum, Giardia intestinalis, Gnathostoma spp, GSS prion, Guanarito virus, Haemophilus ducreyi, Haemophilus influenza, Helicobacter pylon, Henipavirus (Hendra virus Nipah virus), hepatitis A virus, hepatitis B virus (HBV), hepatitis C Virus (HCV), hepatitis D virus, hepatitis E virus, herpes simplex virus 1 and 2 (HSV-1 and HSV-2), Histoplasma capsulatum, human immunodeficiency virus (HIV), Hortaea werneckii, human bocavirus (HBoV), human herpesvirus 6 (HHV-6) and human herpesvirus 7 (HHV-7), human metapneumovirus (hMPV), human papillomavirus (HPV), human parainfluenza viruses (HPIV), influenza virus, Japanese encephalitis virus, JC virus, Junin virus, Kingella kingae, Klebsiella granulomatis, kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, measles virus, Metagonimus yokagawai, Microsporidia phylum, Molluscum contagiosum virus (MCV), mumps virus, Mycobacterium leprae and Mycobacterium lepromatosis, Mycobacterium tuberculosis, Mycobacterium ulcerans, Mycoplasma pneumoniae, Naegleria fowleri, Necator amerimayus, Neisseria gonorrhoeae, Neisseria meningitides, Nocardia asteroids, Nocardia spp, Onchocerca volvulus, Orientia tsutsugamushi, Orthomyxoviridae family (influenza), Paracoccidioides brasiliensis, Paragonimus spp, Paragonimus westermani, parvovirus B19, Pasteurella genus, Plasmodium genus, Pneumocystis jirovecii, Poliovirus, rabies virus, respiratory syncytial virus (RSV), rhinovirus, Rickettsia akari, Rickettsia genus, Rickettsia prowazekii, Rickettsia rickettsia, Rickettsia typhi, rift valley fever virus, rotavirus, rubella virus, sabia virus, Salmonella genus, Sarcoptes scabiei, SARS coronavirus, Schistosoma genus, severe fever thrombocytopenia syndrome virus (SFTSV), Shigella genus, sin nombre virus, hantavirus, Sporothrix schenckii, Staphylococcus genus, Streptococcus agalactiae, pneumoniae such as Streptococcus pneumoniae, Streptococcus pyogenes, Strongyloides stercoralis, Taenia genus, Taenia solium, tickborne encephalitis virus (TBEV), Toxocara mayis, or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, varicella zoster virus (VZV), variola major or variola minor virus, vCJD prion, Venezuelan equine encephalitis virus, Vibrio cholera, west nile virus, western equine encephalitis virus, Wuchereria bancrofti, yellow fever virus, Yersinia enterocolitica, Yersinia pestis, and Yersinia pseudotuberculosis, but is not limited thereto.

The target nucleic acid molecule to which the oligo-nucleotide 122 specifically binds may be derived from cancer or tumor. In an exemplary aspect, the target nucleic acid molecule derived from cancer or tumor may be a nucleic acid molecule specific for cancer or tumor, or a nucleic acid molecule that is a factor or a marker indicating whether or not an onset of cancer or tumor occurred.

In an example, the cancer or tumor includes, but is not limited to, stomach cancer, lung cancer, liver cancer, colorectal cancer, small intestine cancer, skin cancer, pancreatic cancer, and prostate cancer.

In an optional aspect, the target nucleic acid molecule derived from cancer or tumor may be a nucleic acid molecule encoding a protein or a peptide that is a tumor antigen or a fragment thereof, a variant or a derivative thereof.

In an example, the target nucleic acid molecule derived from cancer or tumor may consist of a nucleotide sequence or a transcript sequence thereof, encoding 5T4, 707-AP, 9D7, AFP, AlbZIP HPG1, alpha-5-beta-1-integrin, alpha-5-beta-6-integrin, alpha-actinin-4/m, alpha-methylacyl-coenzyme A racemase, ART-4, ARTC1/m, B7H4, BAGE-1, BCL-2, bcr/abl, beta-catenin/m, BING-4, BRCA1/m, BRCA2/m, CA 15-3/CA 27-29, CA 19-9, CA72-4, CA125, calreticulin, CAMEL, CASP-8/m, cathepsin B, cathepsin L, CD19, CD20, CD22, CD25, CDE30, CD33, CD4, CD52, CD55, CD56, CD80, CDC27/m, CDK4/m, CDKN2A/m, CEA, CLCA2, CML28, CML66, COA-1/m, coactosin-like protein, collage XXIII, COX-2, CT-9/BRD6, Cten, cyclin B1, cyclin D1, cyp-B, CYPB1, DAM-10, DAM-6, DEKmay, EFTUD2/m, EGFR, ELF2/m, EMMPRIN, EpCam, EphA2, EphA3, ErbB3, ETV6-AML1, EZH2, FGF-5, FN, Frau-1, G250, GAGE-1, GAGE-2, GAGE-3, GAGE-4, GAGE-5, GAGE-6, GAGE7b, GAGE-8, GDEP, GnT-V, gp100, GPC3, GPNMB/m, HAGE, HAST-2, hepsin, Her2/neu, HERV-K-MEL, HLA-A*0201-R171, HLA-A11/m, HLA-A2/m, HNE, homeobox NKX3.1, HOM-TES-14/SCP-1, HOM-TES-85, HPV-L1, HPV-E6, HPV-E7, HSP70-2M, HST-2, hTERT, iCE, IGF-1R, IL-13Ra2, IL-2R, IL-5, immature laminin receptor, kallikrein-2, krein-4, Ki67, KIAA0205, KIAA0205/m, KK-LC-1, K-Ras/m, LAGE-A1, LDLR-FUT, MAGE-A1, MAGE-A2, MAGE-A3, MAGE-A4, MAGE-A6, MAGE-A9, MAGE-A10, MAGE-A12, MAGE-B1, MAGE-B2, MAGE-B3, MAGE-B4, MAGE-B5, MAGE-B6, MAGE-B10, MAGE-B16, MAGE-B17, MAGE-C1, MAGE-C2, MAGE-C3, MAGE-D1, MAGE-D2, MAGE-D4, MAGE-E1, MAGE-E2, MAGE-F1, MAGE-H1, MAGEL2, mammaglobin A, MART-1/Melan-A, MART-2, MART-2/m, matrix protein 22, MC1R, M-CSF, ME1/m, mesothelin, MG50/PXDN, MMP11, MN/CA IX-antigen, MRP-3, MUC-1, MUC-2, MUM-1/m, MUM-2/m, MUM-3/m, myosin class I/m, NA88-A, N-acetylglucosaminyl transferase-V, neo-PAP, neo-PAP/m, NFYC/m, NGEP, NMP22, NPM/ALK, N-Ras/m, NSE, NY-ESO-1, NY-ESO-B, OA1, OFA-iLRP, OGT, OGT/m, OS-9, OS-9/m, osteocalcin, osteopontin, p15, p190 minor bcr-abl, p53, p53/m, PAGE-4, PAI-1, PAI-2, PAP, PART-1, PATE, PDEF, Pim-1-kinase, Pin-1, Pml/PAR alpha, POTE, PRAME, PRDX5/m, prostein, proteinase-3, PSA, PSCA, PSGR, PSM, PSMA, PTPRK/m, RAGE-1, RBAF600/m, RHAMM/CD168, RU1, RU2, S-100, SAGE, SART-1, SART-2, SART-3, SCC, SIRT2/m, Sp17, SSX-1, SSX-2/HOM-MEL-40, SSX-4, STAMP-1, STEAP-1, survivin, survivin-2B, SYT-SSX-1, SYT-SSX-2, TA-90, TAG-72, TARP, TEL-AML1, TGF beta, TGF betaRII, TGM-4, TPI/m, TRAG-3, TRG, TRP-1, TRP-2/6b, TRP/INT2, TRP-p8, tyrosinase, UPA, VEGFR1, VEGFR-2/FLK-1, WT1 and immunoglobulin genotype of lymphocytes or T cell receptor genotype of lymphocytes, fragments thereof, variants or derivatives thereof, but is not limited thereto.

The target nucleic acid molecule may be cell-free DNA (cfDNA). The term “cfDNA”, used herein, refers to a fragment of DNA that does not exist in the cell nucleus and floats in the blood. The cfDNA may be derived from cancer cells or pathogens. In addition, cfDNA derived from tumor cells or pathogens may be found in body fluids such as blood, plasma, or urine.

The oligo-nucleotide may be one having the 5′ end or the 3′ end modified with a thiol group, and is capable of binding to a maleimide group of the bead by the thiol group.

The oligo-nucleotide may consist of 20 to 100 nucleotides.

The oligo-nucleotide may be a primer.

The term “primer”, used herein, refers to an oligo-nucleotide that hybridizes to complementary RNA or DNA target polynucleotide, and for example, functions as a starting point for stepwise synthesis of polynucleotides from mononucleotides by an action of a nucleotidyltransferase, which is generated in a polymerase chain reaction. Primers used in the present disclosure may include naturally occurring dNMP (that is, dAMP, dGMP, dCMP, and dTMP), modified nucleotides, or non-natural nucleotides. In addition, primers may also include ribonucleotides. The primer may be one that complementarily binds to cell-free nucleic acid.

Another aspect provides a method of preparing a bead complex including:
(a) transforming the surface of the bead into a structure of Formula 3, which is modified with an amino group, by reacting polyethyleneimine (PEI) and the bead having a surface to which an epoxy group represented by Formula 2 is connected;
(b) transforming the surface of the bead into a structure of Formula 5, which is modified with a maleimide group, by reacting carboxylic acid of Formula 4 with the bead having the structure of Formula 3, which is modified with an amino group; and
(c) conjugating by reacting a bead having the structure of Formula 5, which is modified with a maleimide group, with an oligo-nucleotide that binds specifically to a target nucleic acid molecule and has an end modified with aliphatic thiol of Formula 6 below:

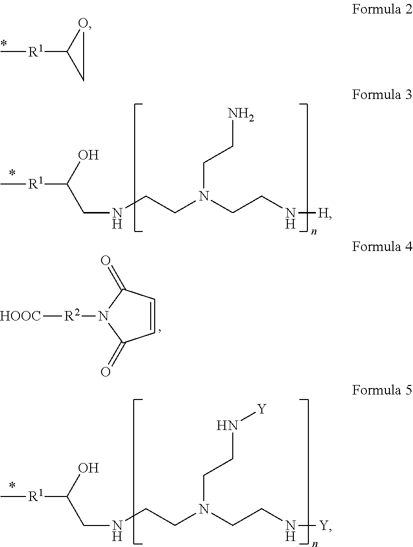

wherein in Formula 5, Y is hydrogen or

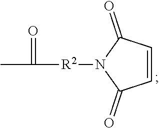

and at least one Y is

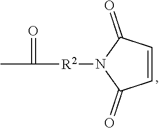

HS—R³—* Formula 6
wherein in Formulas 2 to 6, R¹, R², R³, n, and an asterisk are each the same as defined in claim 1.

In an embodiment, PEI of process (a) may be reacted by being mixed in an amount of 0.1 parts by weight to 1.5 parts by weight, preferably 0.3 parts by weight to 1.2 parts by weight, or more preferably 0.5 parts by weight to 1.0 part by weight, with respect to 1 part by weight of the bead having the surface to which an epoxy group connected.

In an embodiment, process (a) may be performed for 12 hours to 24 hours.

PEI may have a molecular weight of 1,000 to 100,000, preferably 10,000 to 70,000, or more preferably 20,000 to 50,000.

PEI may be linear or branched PEI.

In an embodiment, the carboxylic acid of Formula 4 is not particularly limited, and may be at least one selected from the group consisting of 3-maleimidopropanoic acid, 6-maleimidohexanoic acid, and 11-maleimidodecanoic acid. Preferably, the carboxylic acid of Formula 4 may be 6-maleimidohexanoic acid.

In another aspect, a method is disclosed as a method of preparing the above-described bead complex, including: transforming a surface of a nano-bead with a linker having a structure of Formula 10, by reacting the nano-bead, to which surface an amino group represented by Formula 8 below is connected, and a carboxylic acid represented by Formula 9; and reacting the nano-bead, of which surface is modified with the linker having the structure of Formula 10, with an oligo-nucleotide that binds specifically to a target nucleic acid molecule and has an end modified with aliphatic thiol of Formula 11.

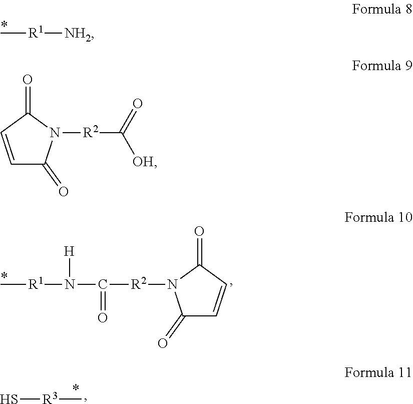

(in Formulas 8 to 11, R¹, R², R³, and the asterisks are each the same as defined in claim 17.)

Another aspect provides a use of the bead complex for detection of a target nucleic acid molecule.

Another aspect provides an assay kit for detecting a target nucleic acid molecule in a biological sample, including the bead complex.

The biological sample may include urine, saliva, sputum, blood and nasopharyngeal smear.

In an embodiment, the assay kit may be an electrophoresis kit, a next generation sequencing kit, or a nucleic acid amplification kit each including: a target nucleic acid molecule that is bound specifically to an oligo-nucleotide constituting a bead complex, a nucleic acid polymerase, and a buffer for nucleic acid polymerization.

The kit may be a nucleic acid amplification kit including a target nucleic acid molecule that is bound specifically to an oligo-nucleotide constituting the bead complex, a nucleic acid polymerase, and a buffer for nucleic acid amplification.

The buffer for nucleic acid amplification may further include: deoxyribonucleotide triphosphate (dNTP) and/or nucleotide triphosphate (NTP), probes labeled with a fluorescent material and/or radioactive isotope for detection of presence of amplified products generated by nucleic acid amplification, and functional additives such as a polymerase stabilizer, in addition to the bead complex and the nucleic acid polymerase.

The polymerase stabilizer may include: bovine serum albumin (BSA), or a polysaccharide selected from the group consisting of cellulose, hydroxypropyl cellulose, hydroxypropyl methylcellulose, glycosaminoglymay, pullulan, alginic acid, carrageenan, aribinogalactan, hemicellulose, dextran, chitosan, glycol chitosan, starch, and combinations thereof. An amount of the polymerase stabilizer included in the buffer for nucleic acid amplification may be 0.01% (w/v) to 10% (w/v), for example, 0.5% (w/v) to 5% (w/v) or 0.5% (w/v) to 3% (w/v). When an amount of the polymerase stabilizer satisfies the above-mentioned range, nucleic acid amplification may be performed efficiently.

The term “polymerase”, used herein, generally refers to a substance that catalyzes polymerization. Polymerases may be used to extend nucleic acid primers paired with a template strand by introduction of nucleotides or nucleotide analogues. Polymerases are capable of adding new strands of DNA by extending the 3′ end of the existing nucleotide chain, by adding new nucleotides corresponding to the template strand one at a time through generation of phosphodiester bonds.

These nucleic acid polymerases are not particularly limited, within the range that amplification of the target nucleic acid molecule may be performed using the polymerase. More specifically, the polymerase includes a DNA polymerase, an RNA polymerase, a thermostable polymerase, a wild-type polymerase, and a modified polymerase. More specifically, the polymerase includes, but is not limited to, a “Klenow fragment” of E. coli DNA polymerase I, a bacteriophage T7 DNA polymerase, a bacteriophage T4 DNA polymerase, a 029 (phi29) DNA polymerase, a Taq polymerase, a Tth polymerase, a Tli polymerase, a Pfu polymerase, a Pwo polymerase, a VENT polymerase, a DEEPVENT polymerase, an EXTaq polymerase, an LA-Taq polymerase, an Sso polymerase, a Poc polymerase, a Pab polymerase, an Mth polymerase, an ES4 polymerase, a Tru polymerase, a Tac polymerase, a Tne polymerase, a Tma polymerase, a Tea polymerase, a Tih polymerase, a Tfi polymerase, a platinum Taq polymerase, a Tbr polymerase, a Tfl polymerase, a Pfutubo polymerase, a Pyrobest polymerase, a Pwo polymerase, a KOD polymerase, a Bst polymerase, a Sac polymerase, a polymerase having 3′ to 5′ exonuclease activity, and variants and derivatives thereof. In an example, the polymerase is a thermostable DNA polymerase that may be obtained from various bacterial species, such as Thermus aquaticus (Taq), Thermus thermophilus (Tth), Thermus filiformis, Thermis flavus, Thermococcus literalis and/or Pyrococcus furiosus (Pfu), but is not limited thereto.

In an embodiment, for the polymerase, dNTP and/or NTP in the buffer for nucleic acid amplification, a commercialized product in a form of a premix may be used. In an embodiment, the nucleic acid polymerase and dNTP (and/or NTP) may be included in an amount of 5% (w/v) to 40% (w/v), for example 10% (w/v) to 33% (w/v) or 15% (w/v) to 25% (w/v) in a buffer for nucleic acid amplification.

The buffer for nucleic acid amplification may include a dNTP mixture (dATP, dCTP, dGTP, dTTP) and/or an NTP mixture (ATP, CTP, GTP, TTP), other additives for the nucleic acid amplification, and nucleic acid polymerase cofactors. When performing nucleic acid amplification, it may be desirable to supply components necessary for the reaction in excess in the reaction vessel. The excess amounts of the components required for nucleic acid amplification refer to amounts such that the amplification is not substantially limited by the concentration of the components. Cofactors, such as Mg²⁺, and dNTP may be supplied to the reaction solution to such an extent that a desired amplification may be achieved.

For example, in nucleic acid amplification, annealing is performed under stringent conditions that allow specific binding between a nucleotide sequence of a target nucleic acid molecule and a primer sequence. The term “annealing” or “priming” refers to apposition of an oligo-nucleotide or a nucleic acid to a template nucleic acid molecule, whereby a polymerase polymerizes nucleotides to form a complementary nucleic acid molecule to the template nucleic acid molecule or a fragment thereof. Stringent conditions for annealing are sequence-dependent and vary according to environmental variables. In an exemplary aspect, the bead complex according to the present disclosure in a buffer for nucleic acid amplification may be contained in a concentration of 5% (w/v) to 40% (w/v), for example, 5% (w/v) to 30% (w/v), or 5% (w/v) to 20% (w/v).

When necessary, a buffer for nucleic acid amplification may further include an additive for nucleic acid amplification. For example, the additive for nucleic acid amplification may be selected from the group consisting of mannitol, polyethylene glycol (for example, PEG 10,000), trehalose, betaine, and combinations thereof. In this regard, the additive for nucleic acid amplification may be added in a concentration of 0.5% (w/v) to 30% (w/v), for example, 0.5% (w/v) to 20% (w/v) or 1% (w/v) to 15% (w/v) to the buffer for nucleic acid amplification.

The buffer for nucleic acid amplification may include an appropriate buffer for nucleic acid amplification. The type of the buffer is not particularly limited, but may be selected from the group consisting of organic acids, glycine, histidine, glutamate, succinate, phosphate, acetate, citrate, tris (for example, tris-EDTA), hydroxyethyl piperazine ethane sulfonic acid (HEPES), amino acids and combinations thereof.

The amplified target nucleic acid molecule may be labeled with a detectable labeling material. In an exemplary aspect, the labeling material may be a material that emits fluorescence, phosphorescence, chemiluminescence, or radioactivity, but is not limited thereto. For example, the labeling material may be fluorescein, phycoerythrin, rhodamine, lissamine, Cy-5 or Cy-3.

When amplifying a target nucleic acid molecule, by labeling Cy-5 or Cy-3 at the 5′ end and/or the 3′ end of the oligo-nucleotide, and performing nucleic acid amplification (for example, real-time polymerase chain reaction), the target nucleic acid may be labeled with a detectable fluorescent labeling material. In addition, the labeling using a radioactive material may be performed by adding radioactive isotopes such as P³² and/or S¹⁵ to the buffer for nucleic acid amplification according to the present disclosure when performing nucleic acid amplification (for example, real-time polymerase chain reaction), so that as amplification products are synthesized, radioactivity is incorporated into the amplification product, and the amplification product may be radioactively labeled.

For labeling, various methods commonly practiced in the art to which the present disclosure pertains may be used. For example, labeling may be performed by using a nick translation method, a random priming method (Multiprime DNA labelling systems booklet, “Amersham” (1989)), and a kination method (Maxam & Gilbert, Methods in Enzymology, 65:499 (1986)). The label provides a signal detectable by measurements of fluorescence, radioactivity, and phosphorescence, chromometry, gravimetric measurement, X-ray diffraction or absorption, measurement of magnetism, measurement of enzymatic activity, mass analysis, measurement of binding affinity, hybridization high-frequency, or nano-crystal.

With respect to nucleic acid amplification, for example, polymerase chain reaction (PCR), denaturation for separation of a target nucleic acid molecule and an oligo-nucleotide double chain, annealing and polymerization may be appropriately adjusted and controlled, depending on the composition and length of the oligo-nucleotide 122 and the target nucleic acid molecule binding specifically thereto.

The nucleic acid amplification is not particularly limited within the range that the nucleic acid amplification is capable of amplifying a target nucleic acid molecule that may be present in a biological sample. For example, the nucleic acid amplification may use any method selected from the group consisting of polymerase chain reaction (PCR), reverse transcription-polymerase chain reaction (RT-PCR), real-time polymerase chain reaction (teal-time PCR), reverse transcription, complementary DNA synthesis, repair chain reaction, multiplex PCR, transcription-mediated amplification (TMA), self-sustained sequence replication, selective amplification of target polynucleotide sequences, consensus sequence primed polymerase chain reaction (CP-PCR), arbitrarily primed polymerase chain reaction (AP-PCR), loop-mediated isothermal amplification (LAMP), real-time nucleic acid sequence-based amplification (NASBA), strand displacement amplification (SDA), multiple displacement amplification (MDA), rolling circle amplification (RCA), ligase chain reaction (LCR), helicase dependent amplification (HDA), ramification-extension amplification method (RAM), in vitro transcription-based amplification system (TAS), and combinations thereof, but is not limited thereto.

In an embodiment, an assay kit for detecting the binding of the oligo-nucleotide constituting the bead complex and the target nucleic acid molecule present in the biological sample may be an electrophoresis kit or a next generation sequencing (NGS) kit, but is not limited thereto.

Another aspect provides a method of detecting a target nucleic acid molecule in a biological sample, including: reacting the bead complex with a biological sample;
separating the bead complex in which the target nucleic acid present in the biological sample and the oligo-nucleotide are bound; and
detecting whether the oligo-nucleotide and the target nucleic acid molecule are bound.

For example, the process of detecting whether the oligo-nucleotide and the target nucleic acid molecule are bound may include performing nucleic acid amplification using the target nucleic acid molecule bound to the oligo-nucleotide as a template.

Optionally, the process of separating the bead complex, in which the target nucleic acid molecule present in the biological sample and the oligo-nucleotide are bound, may be performed by using a magnet.

The biological sample may include, but is not limited to, urine, saliva, sputum, blood, and nasopharyngeal smear. When nucleic acid molecules present in the biological sample are in the form of DNA, the nucleic acid molecules in the biological sample may be transformed into single-stranded nucleic acid molecules before the biological sample and the bead complex react.

When necessary, before reacting the bead complex with the biological sample, the method may further include transforming nucleic acid molecules present in the biological sample into single-stranded nucleic acid molecules.

For example, the transformation into single-stranded nucleic acid molecules may be performed by applying heat to the biological sample, in which case, the heat treatment may be performed at 70° C. to 100° C. for 2 minutes to 10 minutes, but is not limited thereto.

In addition to the target nucleic acid molecule, various non-target nucleic acid molecules may be present in a biological sample. An oligo-nucleotide capable of hybridizing with a target nucleic acid molecule is bound to the outer surface of the bead complex according to the present disclosure. Therefore, when the bead complex according to the present disclosure is reacted with a biological sample in which target nucleic acid molecules are present, only the target nucleic acid molecules present in the biological sample bind specifically to the oligo-nucleotide constituting the bead complex according to the present disclosure. On the other hand, non-target nucleic acid molecules present in the biological sample do not bind to the bead complex according to the present disclosure, and remain as free nucleic acid molecules in the biological sample.

In an embodiment, when the beads according to the present disclosure are made of magnetized beads, the bead complexes, in which a target nucleic acid and an oligo-nucleotide are bound, may be separated from non-target nucleic acid molecules remaining in a biological sample by using a magnet.

In an embodiment, the process of detecting whether the oligo-nucleotide and the target nucleic acid molecule are bound may be performing by polymerase chain reaction (PCR) using the target nucleic acid molecule bound to the oligo-nucleotide as a template.

The polymerase chain reaction may be real-time polymerase chain reaction, but is not limited thereto. With respect to the polymerase chain reaction, denaturation for separation of the target nucleic acid molecule and the oligo-nucleotide double strand, annealing, and polymerization may be appropriately adjusted and controlled, depending on the composition and length of the oligo-nucleotide and the target nucleic acid molecule binding specifically thereto.

In another aspect, a method of purifying a target nucleic acid molecule is provided, the method including: reacting the above-described bead complex with a biological sample; and separating the bead complex in which the target nucleic acid molecule present in the biological sample and the oligo-nucleotide are bound.

For example, the process of separating the bead complex, in which the target nucleic acid molecule present in the biological sample and the oligo-nucleotide are bound, may be performed by using a magnet.

The term “epoxy bead”, used herein, refers to a bead having the surface to which an epoxy group is connected.

The term “epoxy-PEI bead”, used herein, refers to a bead, in which the surface of the bead is coated with PEI containing an amino group, by reacting the epoxy bead with polyethyleneimine (PEI).

The term “epoxy-PEI-maleimide bead”, used herein, refers to a bead, in which the surface of the bead is modified with a maleimide group by reacting the amino group on the surface of the epoxy-PEI bead with a carboxyl group of a compound having a maleimide group and a carboxyl group at both ends.

The term “bead complex”, used herein, refers to an epoxy-PEI-maleimide bead and a bead including an oligo-nucleotide that binds specifically to a target nucleic acid molecule, and binds to the bead surface with one end conjugated with the maleimide group.

The terms “polynucleotide” and “nucleic acid molecule”, used herein, are used interchangeably, and refer to a polymer of nucleotides of any length and include DNA (for example, cDNA) and RNA molecules generically. “Nucleotide”, the building block of a nucleic acid molecule, refers to deoxyribonucleotides, ribonucleotides, modified nucleotides or bases, and/or analogs thereof, or any substrate that may be incorporated into polymers by DNA or RNA polymerases, or by synthetic reactions. Polynucleotides may include modified nucleotides, and analogs, in which sugars or bases are modified, such as methylated nucleotides and analogs thereof.

There are mutations in nucleotides that do not result in mutations in proteins. Such nucleic acids include nucleic acid molecules including functionally equivalent codons or codons encoding the same amino acid (e.g., due to codon degeneracy, there are six codons for arginine (Arg) or serine (Ser)), or a codon encoding a biologically equivalent amino acid.

In addition, mutations in nucleotides may cause changes in the protein itself. Even when a mutation causes a change in the amino acid of the protein, a protein exhibiting almost the same activity as the protein of the present disclosure may be obtained.

To the extent that a nucleic acid molecule or a polynucleotide has characteristics of the nucleic acid molecule or polynucleotide of the present disclosure, it is clear to those skilled in the art that the peptides and nucleic acid molecules of the present disclosure are not limited to the amino acid sequence or base sequence described in the sequence list.

Meanwhile, the term “amino acid”, used herein, is used in the broadest sense and is intended to include naturally-occurring L-amino acids or residues. One-letter abbreviations and/or three-letter abbreviations commonly used for naturally-occurring amino acids may be used herein.

Amino acids not only include D-amino acids but also chemically-modified amino acids, such as amino acid analogs, naturally-occurring amino acids normally not incorporated into proteins, such as norleucine, or any chemically-synthesized compounds having properties of amino acids known in the art to which the present disclosure pertains. For example, included within the definition of an amino acid are analogs or mimetics of phenylalanine or proline that allow conformational restriction of a peptide compound, which is the same with natural phenylalanine or proline. Such analogs and mimetics may be referred to herein as “functional equivalents” of amino acids. Other examples of amino acids are listed in a reference (Roberts and Velaccio, The Peptides: Analysis, Synthesis, Biology, Eds. Gross and Meiehofer, Vol. 5, p. 341 (Academic Press, Inc.: N. 1983).

For example, synthetic peptides synthesized by standard solid-phase synthesis techniques are not limited to the amino acids encoded by the gene, and thus allow for a wider variety of substitutions for a given amino acid. Amino acids not encoded by the genetic code may be referred to herein as “amino acid analogs”. For example, amino acid analogs include 2-aminoadipic acid (Aad) for Glu and Asp; 2-aminopimelic acid (Apm) for Glu and Asp; 2-aminobutyric acid (Abu) for Met, Leu and other aliphatic amino acids; 2-aminoheptanoic acid (Ahe) for Met, Leu and other aliphatic amino acids; 2-aminobutyric acid for Gly (Aib); cyclohexylalanine (Cha) for Val, Leu and IIe; homoarginine (Har) for Arg and Lys; 2,3-diaminopropionic acid (Dap) for Lys, Arg and His; N-ethylglycine (EtGly) for Gly, Pro and Ala; N-ethylglycine (EtGly) for Gly, Pro and Ala; N-ethylasparagine (EtAsn) for Asn and Gin; hydroxylysine (Hyl) for Lys; allohydroxylysine (AHyl) for Lys; 3-(and 4-)hydroxyproline for Pro, Ser and Thr (3Hyp, 4Hyp); allo-isoleucine (Alle) for IIe, Leu and Val; 4-amidinophenylalanine for Arg; N-methylglycine (MeGly, sarcosine) for Gly, Pro and Ala; N-methylisoleucine for IIe (Melle); norvaline (Nva) for Met and other aliphatic amino acids; norleucine (NIe) for Met and other aliphatic amino acids; ornithine (Orn) for Lys, Arg and His; citrulline (Cit) and methionine sulfoxide (MSO) for Thr, Asn and Gln; and N-methylphenylalanine (MePhe) for Phe, trimethylphenylalanine, halo-(F-, Cl-, Br- or I-)phenylalanine or trifluorylphenylalanine.

The term “peptide”, used herein, includes all proteins, protein fragments, and peptides isolated from naturally occurring ones or ones synthesized chemically or by recombinant technique.

In an aspect, peptide variants, for example, peptide variants having at least one amino acid substitution are provided. The term “peptide variant”, used herein, refers to one having one or more amino acids having substitutions, deletions, additions and/or insertions in the amino acid sequence of a peptide, and refers to one having almost the same biological function as the peptide composed of original amino acids. The peptide variant has to have identity of at least 70%, preferably, at least 90%, or more preferably, at least 95% to the original peptide.

Such peptide variants may include amino acid variants known as “conservative”. Variants may also include non-conservative changes. In an exemplary aspect, a sequence of the variant polypeptide differs from the original sequence by substitution, deletion, addition, or insertion of five or less amino acids. Variants may also be altered by deletion or addition of amino acids that have minimal effect on the immunogenicity, secondary structure and hydropathic nature of the peptide.

“Conservative” substitution means that there is no significant change in properties such as secondary structure and hydropathic nature of a polypeptide even when one amino acid is substituted with another amino acid. Amino acid variations may be obtained based on relative similarity of amino acid side chain substituents, such as similarity of polarity, charge, solubility, hydrophobicity, hydrophilicity and/or amphipathic nature, etc. In an example, amino acid exchanges in proteins or peptides that do not entirely alter activity of a molecule are known in the art (H. Neurath, RLHill, The Proteins, Academic Press, New York, 1979). The most common exchanges are amino exchanges between acid residues Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, and Asp/Gly.

In consideration of the above-described variation having biologically equivalent activity, it is construed that the nucleic acid molecule encoding the peptide and/or protein according to the present disclosure includes sequences exhibiting substantial identity to the sequence described in the sequence list. The substantial identity means a sequence showing homology of at least 61%, more preferably. homology of at least 70%, even more preferably, homology of at least 80%, and most preferably, homology of at least 90%, when the sequence of the present disclosure and any other sequence are aligned to the maximum extent and the aligned sequences are analyzed by using an algorithm commonly used in the art. Alignment methods for sequence comparison are known in the art.

The term “hybridization under stringent condition”, used herein, means that two single-stranded nucleic acid molecules consist of complementary nucleotides of at least 70%, for example, 80% or more, or 90% or more.

For example, an oligo-nucleotide 122 that binds specifically to a target nucleic acid molecule may be a probe or a primer. “Primer”, used herein, generally refers to a nucleic acid molecule that is complementary to a portion of a template nucleic acid molecule.

In an example, a primer may be complementary to a portion of a strand of a template nucleic acid molecule. The primer may be a strand of nucleic acid for synthesis of a target nucleic acid molecule in nucleic acid amplification, in which case, the primer may hybridize to the template strand, and the nucleotide may be added to the end(s) of the primer with the help of a polymerase. Thus, while the target nucleic acid molecule is being replicated, the polymerase catalyzing the replication of the target nucleic acid molecule initiates replication at the 3′ end of the primer attached to the target nucleic acid molecule, thereby synthesizing and replicating the opposite strand of the target nucleic acid molecule.

In an optional aspect, A primer may be fully or partially complementary to the template nucleic acid molecule. A primer may exhibit sequence identity or homology or complementarity to a template nucleic acid. Homology or sequence identity or complementarity between the primer and the template nucleic acid may be based on the length of the primer. For example, when a primer consists of about 20 nucleotides, the primer may include 10 or more adjacent nucleic acid bases complementary to the template nucleic acid.

On the other hand, the oligo-nucleotide 122 may bind specifically to a target nucleic acid molecule present in a biological sample.

In an exemplary aspect, the oligo-nucleotide may consist of 20 to 100 nucleotides. When 20 to 100 nucleotides constitute the oligo-nucleotide, the oligo-nucleotide is capable of hybridizing with a target nucleic acid molecule present in a biological sample under stringent conditions. Accordingly, it is possible to isolate and purify target nucleic acid molecules specific for the oligo-nucleotide with high sensitivity.

### Advantageous Effects of Disclosure

According to an aspect, the bead complex may effectively isolate, extract, and detect a target nucleic acid molecule in a biological sample, and by analyzing whether the target nucleic acid molecule is present in the biological sample, sensitivity of the analysis may be improved.

In addition, by introducing a linker having a predetermined length between the bead and the oligo-nucleotide, steric hindrance in the molecule may be minimized, non-specific binding between the linker and the base of the oligo-nucleotide may be inhibited, and specific binding between the nano-bead and the linker may be implemented.

### BRIEF DESCRIPTION OF DRAWINGS

FIGS. 1A to 1B are schematic diagrams showing manufacturing processes of a bead complex according to an embodiment.

FIG. 2 is a schematic diagram illustrating a process of collecting target nucleic acid molecules in a biological sample using the bead complex according to an embodiment.

FIGS. 3A to 3B show results of measuring the average particle size of the bead complex according to an embodiment.

FIGS. 4A to 4B show results of measuring the surface charge of the bead according to an embodiment.

FIGS. 5A to 5B show results of confirming through PCR the results of extracting cfDNA using the bead complex according to an embodiment.

FIG. 6 shows results of confirming through electrophoresis the results of cfDNA extraction using the bead complex according to an embodiment.

FIG. 7 is a schematic diagram schematically showing configuration of a bead complex according to an exemplary embodiment of the present disclosure.

FIG. 8 is a schematic diagram schematically illustrating a process of manufacturing the bead complex according to an exemplary embodiment of the present disclosure.

FIG. 9 is a schematic diagram schematically illustrating a process of isolating a target nucleic acid molecule from a biological sample using the bead complex prepared according to an exemplary embodiment of the present disclosure.

FIG. 10 is a graph showing results of measuring the average particle size of the bead complex synthesized according to an exemplary embodiment of the present disclosure using the DLS method.

FIG. 11 is a graph showing results of measuring the average surface charge of the bead complex synthesized according to an example embodiment of the present disclosure by using a Zetasizer.

FIGS. 12A to 12D are graphs each showing PCR analysis results using the bead complex synthesized according to an example embodiment of the present disclosure.

FIG. 13 is photographs showing results of measuring detection of target nucleic acid molecules extracted by using the bead complex synthesized according to an example embodiment of the present disclosure by using electrophoresis.

### DETAILED DESCRIPTION

Currently, in order to find out whether viral disease, bacterial disease, or cancer is present, it is necessary to obtain a biological sample (specimen) such as urine, saliva, sputum, nasopharyngeal smear or blood, and then to extract and purify nucleic acid molecules such as DNA or RNA from inside the biological sample. Various inhibitors, DNA, and RNA exist in a biological sample, and an amount of a nucleic acid molecule to be detected is present in a relatively very low concentration in the entire biological sample.

The currently used nucleic acid extraction method collects all charged substances in a biological sample and then purifies nucleic acid. Next, a test using nucleic acid amplification such as polymerase chain reaction is performed using the purified nucleic acid, and thus, there is a disadvantage of low accuracy and efficiency. In addition, there are disadvantages in that the method of collecting biological samples is invasive and inconvenient and requires help of professional medical staff.

Therefore, the present inventors developed a bead complex capable of detecting only specific target nucleic acid molecules in a biological sample, and rapidly and easily detecting and analyzing with high sensitivity and accuracy whether a virus or a pathogen is infected and/or an onset of cancer has occurred. In order to detect and analyze only a desired specific nucleic acid molecule in a biological sample, bead complexes may be used to collect, purify, and detect the specific nucleic acid molecule, wherein in the bead complex, an oligo-nucleotide, which is capable of specifically reacting with the desired nucleic acid molecule, is conjugated on the surface of a nano-bead.

Hereinafter, preferred embodiments are presented to help understanding of the present disclosure. However, the following embodiments are only provided for easier understanding of the present disclosure, and the contents of the present disclosure are not limited by the following embodiments.

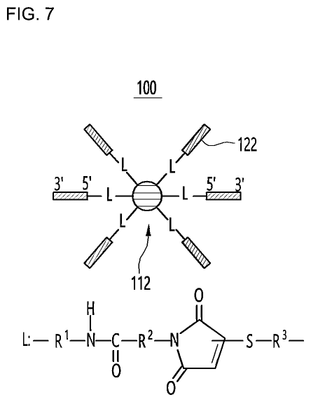

FIG. 7 is a schematic diagram schematically showing configuration of a bead complex according to an exemplary embodiment of the present disclosure. As shown in FIG. 7, a bead complex 100 includes a nano-bead 112 and oligo-nucleotides 122, wherein the oligo-nucleotides 122 have termini conjugated to the surface of a nano-bead 112 through linkers L, and bind specifically to target nucleic acids. The linker L interposed between the surface of the nano-bead 112 and the oligo-nucleotide 122 may have a structure of the following Formula 7:

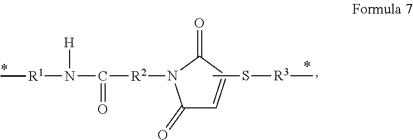

wherein in Formula 7, R¹ is a direct bond or a C₁-C₂₀ aliphatic hydrocarbon group; R² and R³ are each independently a C₃-C₂₀ divalent aliphatic hydrocarbon linker; the asterisk on the left of R¹ indicates a site connected to the surface of the nano-bead, and the asterisk on the right of R³ indicates a site connected to the end of the oligo-nucleotide.

In Formula 7, R¹ may be a direct bond or an aliphatic hydrocarbon group selected from the group consisting of a C₁-C₂₀ alkyl group, a C₂-C₂₀ alkenyl group, a C₂-C₂₀ alkynyl group, and a C₁-C₂₀ alkyl ether group. For example, the aliphatic hydrocarbon group constituting R¹ of Formula 7 may be selected from the group consisting of a C₁-C₁₀ alkyl group, a C₂-C₂₀ alkenyl group, a C₂-C₂₀ alkynyl group, and a C₁-C₂₀ alkyl ether group, but is not limited thereto. In addition, R² and R³ in Formula 7 may each independently be a C₃-C₂₀ alkylene group, for example, a C₃-C₁₀ alkylene group, or a C₅-C₁₀ alkylene group, but is not limited thereto.

The linker L interposed between the nano-bead 112 and the oligo-nucleotide 122 includes R² and R³, which are two aliphatic hydrocarbon linkers each having at least 3 carbons. By introducing linkers L having a predetermined length, the nano-bead 112 constituting a bead complex 100 and the oligo-nucleotides 122 capable of binding specifically to a target nucleic acid molecule are spaced apart with a sufficient distance. Accordingly, intra-molecular and/or inter-molecular steric hindrance between the nano-bead 112 and the oligo-nucleotides 122 in a bead complex 100 may be minimized.

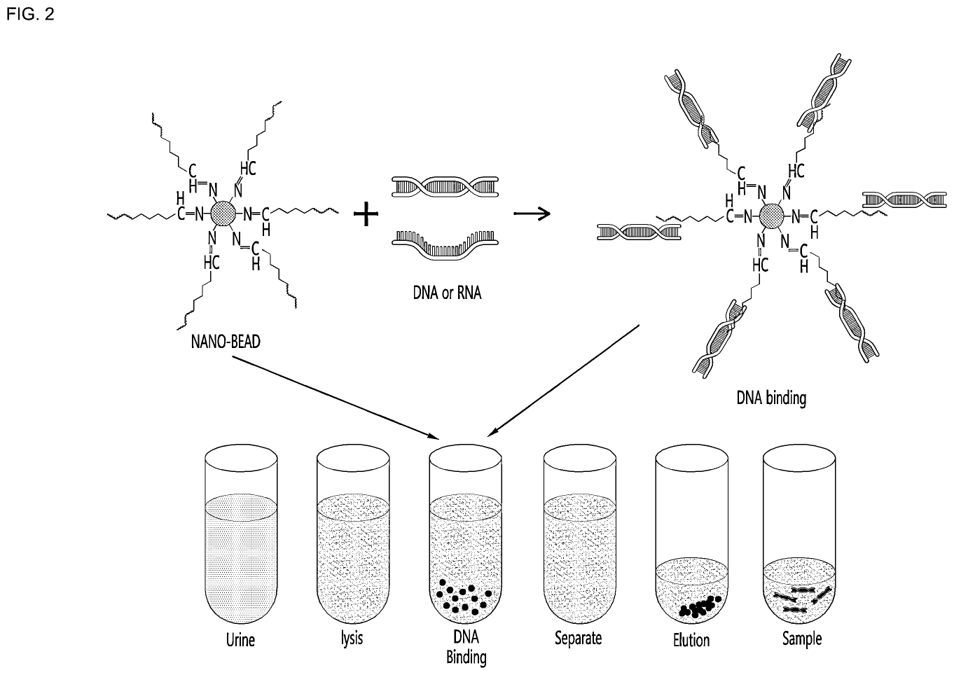

In addition, in the process of synthesizing or manufacturing a bead complex 100, an amide bond may be specifically formed between the amine group (—NH₂), which is a reactive functional group directly or indirectly connected to the surface of the nano-bead 112 constituting the initial bead 110 (see FIG. 2), and the carboxylic acid group (—COOH) applied to form a linker L, and a non-specific binding between the carboxylic acid group and the reactive amine group, present in the base constituting the oligo-nucleotide 122, may be inhibited.

In a bead complex 100, one end of a linker L interposed between the nano-bead 112 and the oligo-nucleotide 122 may be connected to the 5′ end or the 3′ end of the oligo-nucleotide 122. For example, one end of the linker L may be connected to the 5′ end of the oligo-nucleotide 122.

In an exemplary aspect, the nano-beads 112 may be made of an inorganic material. For example, the inorganic material forming the nano-beads 112 may be a non-metal material selected from the group consisting of iron oxide, silica, glass, and combinations thereof and/or gold, silver, copper, combinations thereof, and alloys thereof, but is not limited thereto.

In another exemplary aspect, the nano-beads 112 may be made of an organic material. The organic material may be a polymer material. For example, the organic material forming the nano-beads 112 may be a polymer resin selected from the group consisting of polystyrene, polypropylene, polyethylene, poly acrylamide, combinations thereof, and copolymers thereof and/or a polysaccharide material such as pullulan, pullulan acetate, cellulose, hydroxypropylmethylcellulose, agarose, chitosan, combinations thereof, or copolymers thereof, but is not limited thereto.

In another optional aspect, the nano-beads 112 may be made of a porous material or a paramagnetic material. For example, the nano-beads 112 may be formed of a magnetic material. When the nano-beads 112 are made of a paramagnetic material, as will be described later, a target nucleic acid molecule bound specifically to the bead complex 100 may be easily separated and purified by using a magnet.

In an exemplary aspect, the oligo-nucleotides 120 may consist of 20 to 100, for example, 20 to 50 nucleotides. When the oligo-nucleotide 122 consists of 20 to 100 nucleotides, the oligo-nucleotide 122 is capable of hybridizing with a target nucleic acid molecule present in a biological sample under stringent conditions. Accordingly, a target nucleic acid molecule specific for the oligo-nucleotide 122 may be separated and purified with high sensitivity.

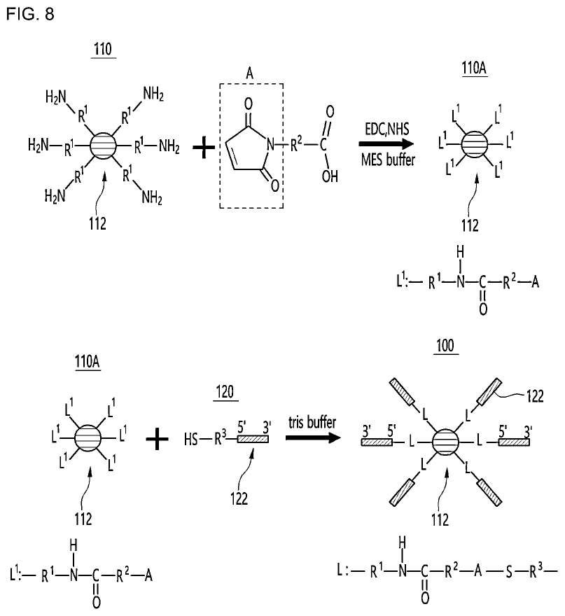

Next, a method of preparing and synthesizing a bead complex according to an example embodiment of the present disclosure will be described with reference to FIG. 8. As shown in the upper left of FIG. 8, a bead intermediate 110A is synthesized by reacting an initial bead 110, in which amine groups represented by the following Formula 8 are connected to the surface of the nano-beads 112, with carboxylic acid having a structure of the following Formula 9, wherein in the bead intermediate, the surface of the nano-bead 112 is modified with linkers (first linker L¹) having a structure of the following Formula 10.

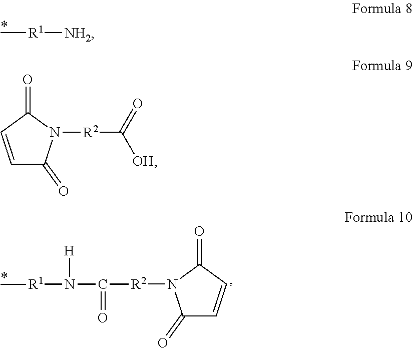

(in Formulas 8 to 10, R¹, R², R³, and an asterisk are each the same as defined in Formula 7.)

In an exemplary aspect, the initial bead 110, in which amine groups having the structure of Formula 8 are connected to the surface of the nano-bead 112, may be activated in a buffer solution adjusted to pH 5 to pH 6. A buffer capable of activating the initial bead 110 may be selected from the group consisting of a 2-[Nmorpholino] ethane sulfonic acid (MES) buffer, a phosphate buffer, sodium acetate, sodium phosphate, and combinations thereof, but is not limited thereto.

On the other hand, an appropriate activator may be used, in order that the amine group, connected to the surface of the nano-bead 112 constituting the initial bead 110, and the carboxylic acid group, constituting a terminus of the aliphatic carboxylic acid having the structure of Formula 8, react to form a stable amide bond. An activator that may be used may be selected from the group consisting of 1-ethyl-3-(3-dimethylaminopropyl)carbodiimide hydrochloride (EDC), N-hydroxysuccinimide (NHS), sulfo-N-hydroxysulfosuccinimide (NHS), 1-cyclohexyl-3-(2-morpholinoethyl)carbodiimide (CMC), dicyclohexyl carbodiimide (DCC), diisopropyl carbodiimide (DIC), and combinations thereof, but is not limited thereto. For example, the activator may be added in a ratio of 10 molar equivalents to 200 molar equivalents, for example, 50 molar equivalents to 150 molar equivalents, with respect to the amine groups connected to the surface of the nano-bead 112 constituting the initial bead 110.

For example, the reaction between the initial bead 110, in which an amine group is connected to the surface of the nano-bead 112, and the aliphatic carboxylic acid having the structure of Formula 8, may be performed at room temperature for 12 hours to 24 hours, but is not limited thereto. When necessary, after the reaction between the initial beads 110 and the aliphatic carboxylic acid is completed, a washing process using the above-described buffer may be performed.

Subsequently, the bead intermediate 110A, in which the surface of the nano-bead 112 is primarily modified or modified with linkers (first linker L¹) having the structure of the Formula 10, is reacted with the oligo-nucleotides 122 having an end of the oligo-nucleotide 122 modified with aliphatic thiol of Formula 11, and a bead complex 100, in which the oligo-nucleotides 122 are connected to the surface of the nano-beads 112 by the final linkers (L) having the structure of Formula 7, is synthesized and prepared.

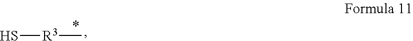

(in Formula 11, R³ and an asterisk are the same as defined in Formula 7.)

The reaction between the bead intermediate 110A and the modified oligo-nucleotides 120 may be carried out at room temperature for 12 hours to 24 hours, for example, at a pH of 7 to 8. When necessary, a reducing agent for activating the terminal thiol groups of the modified oligo-nucleotides 120 may be used before the reaction. For example, a reducing agent for activating the thiol group connected to one end of the oligo-nucleotide 122 may be selected from the group consisting of tris(2-carboxyethyl) phosphine (TCEP), tris(3-hydroxypropyl) phosphine (THPP), dithiothreitol (DTT), 2-mercaptoethanol (ME), 2-mercaptoethylamine (MEA), combinations thereof, and salts thereof (for example, hydrochloride), but is not limited thereto.

When necessary, modified oligo-nucleotides 120 not bound to the surface of the bead intermediate 110A may be removed.

As shown in FIG. 8 and Formula 7, in the finally prepared and synthesized bead complex 100, the linker L interposed between the nano-bead 112 and the oligo-nucleotides 122 includes divalent aliphatic hydrocarbon linkers (R² and R³), each including at least 3 carbon atoms. Accordingly, the nano-bead 112 and the oligo-nucleotides 122 are not directly connected, and are spaced apart from each other at a predetermined distance. Therefore, intramolecular and/or intermolecular steric hindrance between the nano-beads 112 and the oligo-nucleotides 122 constituting the bead complex 100 of the present disclosure may be prevented and minimized.

On the other hand, in the present disclosure, the initial bead 110 having amine groups connected to the surface of the nano-bead 112 is reacted with carboxylic acid having the structure of Formula 9, and a bead intermediate 110A, which has first linkers (L¹) having an amide bond, is synthesized; and the bead intermediate 110A is reacted with the oligo-nucleotides 120 which is modified with aliphatic thiol having the structure of Formula 11 at the end of the oligo-nucleotide 122, to finally synthesize a bead complex 100, in which linkers, each having a predetermined length, are interposed between the nano-bead 112 and the oligo-nucleotides 122.

That is, the amine group, which is a reactive functional group connected to the surface of the nano-bead 112, does not directly react with the carboxylic acid group formed at the end of the oligo-nucleotide, and the amine group connected to the surface of the nano-bead 112 primarily forms an amide bond with the carboxylic acid group of the carboxylic acid having the structure of Formula 8. Accordingly, an amide bond may be specifically formed between the amine group connected to the surface of the nano-bead 112, and the carboxylic acid group of the carboxylic acid having the structure of Formula 8.

According to the present disclosure, the bases constituting the oligo-nucleotides 122 are not directly connected to the nano-bead 112. In the bases constituting the oligo-nucleotides 122 constituting the bead complex 100, steric hindrance due to direct binding to the nano-bead 112 is inhibited. Accordingly, since the oligo-nucleotide 122 efficiently hybridizes with a target nucleic acid molecule, presence or absence of the target nucleic acid molecule in a biological sample may be detected and found by applying the bead complex 100 of the present disclosure.

On the other hand, in case the oligo-nucleotides are conjugated to the surface of the nano-bead, intramolecular and/or intermolecular steric hindrance may be caused between the nano-bead and the oligo-nucleotide. In addition, for example, in case of conjugating so that an amide bond is formed between a carboxylic acid group connected to the surface of the nano-bead and a modified amine group at one end of the oligo-nucleotide, the carboxylic acid group connected to the surface of the nano-bead not only reacts with the amine group located at the one end of the oligo-nucleotide, but also reacts with reactive primary amine groups present in adenine, cytosine, and guanine, among the bases constituting the oligo-nucleotide, to form an amide bond. That is, an amide bond between the nano-bead and the oligo-nucleotide is formed non-specifically.

By the non-specific amide bond between the nano-bead and the bases constituting the oligo-nucleotide, steric hindrance is induced in the bases constituting the oligo-nucleotide, and the oligo-nucleotide including the base, in which steric hindrance is induced, is not capable of specifically hybridizing with the target nucleic acid molecule efficiently. Accordingly, when inducing a direct amide bond between the nano-bead and the oligo-nucleotide, there is a limit in detecting and finding presence or absence of a target nucleic acid molecule in a biological sample due to the modification of the oligo-nucleotide.

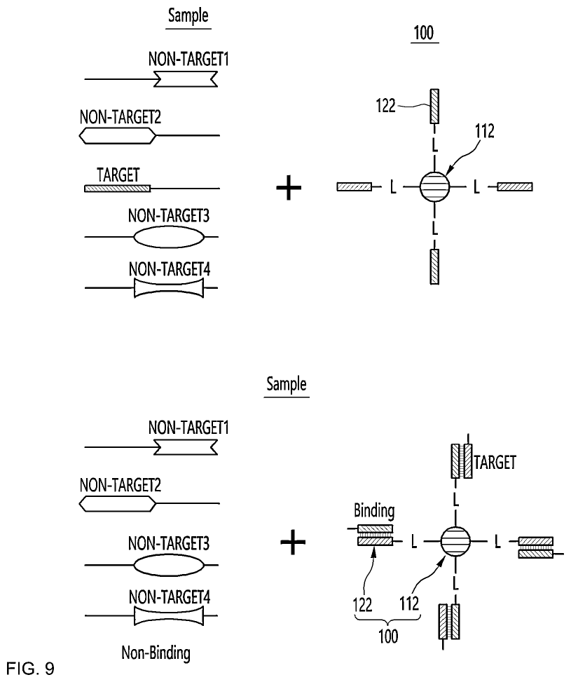

Subsequently, a method of analyzing presence of a target nucleic acid molecule in a biological sample and a method of purifying target nucleic acid molecules in a biological sample using the bead complex according to the present disclosure will be described. FIG. 9 is a schematic diagram schematically illustrating a process of isolating target nucleic acid molecules from a biological sample using the bead complexes prepared according to an exemplary embodiment of the present disclosure. As shown in FIG. 9, the biological sample may be reacted with the above-described bead complex 100, and the bead complex 100, in which the target nucleic acid molecule in the biological sample and the oligo-nucleotide 122 are bound, may be separated, and thus, the target nucleic acid molecules present in a biological sample may be selectively isolated and purified.

For example, biological samples may include, but are not limited to, urine, saliva, sputum, blood, and nasopharyngeal smears. When the nucleic acid molecules present in a biological sample are in the form of DNA, the nucleic acid molecules in the biological sample may be transformed into single-stranded nucleic acid molecules before the biological sample and the bead complex are reacted. For example, the transformation into single-stranded nucleic acid molecules may be performed by applying heat to the biological sample, in which case the heat treatment may be performed at 70° C. to 100° C. for 2 minutes to 10 minutes, but is not limited thereto.

In addition to the target nucleic acid molecule, various non-target nucleic acid molecules may be present in a biological sample. Oligo-nucleotides 122 capable of hybridizing with target nucleic acid molecules are bound to the outer surface of the bead complex 100 according to the present disclosure. Therefore, when the bead complex 100 according to the present disclosure is reacted with a biological sample, in which target nucleic acid molecules are present, only the target nucleic acid molecules present in the biological sample binds specifically to the oligo-nucleotides 122 constituting the bead complex 100 according to the present disclosure. On the other hand, non-target nucleic acid molecules present in the biological sample do not bind to the bead complex 100 according to the present disclosure, and remain as free nucleic acid molecules in the biological sample.

Subsequently, only the target nucleic acid molecules bound specifically to the bead complexes 100 according to the present disclosure may be separated and purified from the biological sample. For example, when the nano-bead 112 is made of a magnetic material, the bead complexes 100 may be separated from the non-target nucleic acid molecules remaining in the sample by using a magnet. Accordingly, from among the nucleic acid molecules present in the biological sample, only the target nucleic acid molecules bound specifically to the bead complexes 100 according to the present disclosure may be separated and purified with high sensitivity.

According to the above-described embodiment, only the target nucleic acid molecules bound to the bead complexes may be specifically purified and separated, and then presence or absence of the target nucleic acid molecule in the biological sample may be analyzed.

On the other hand, the target nucleic acid molecule may bind specifically to the oligo-nucleotide 122 located outside the bead complex 100, which is isolated and purified from the biological sample. In this state, when nucleic acid amplification is performed using the target nucleic acid molecules as templates and the oligo-nucleotides 122 as primers, nucleotides complementary to the target nucleic acid molecule are synthesized at the end of the oligo-nucleotides 122. Accordingly, a plurality of nucleic acid molecules corresponding to the target nucleic acid molecule is amplified, and the presence or absence of the amplification product is identified, and thereby, the presence or absence of the target nucleic acid molecule in the biological sample may be detected and analyzed.

### Example 1. Preparation of Epoxy-PEI-Maleimide Beads and Oligo-Nucleotide-Bead Complexes

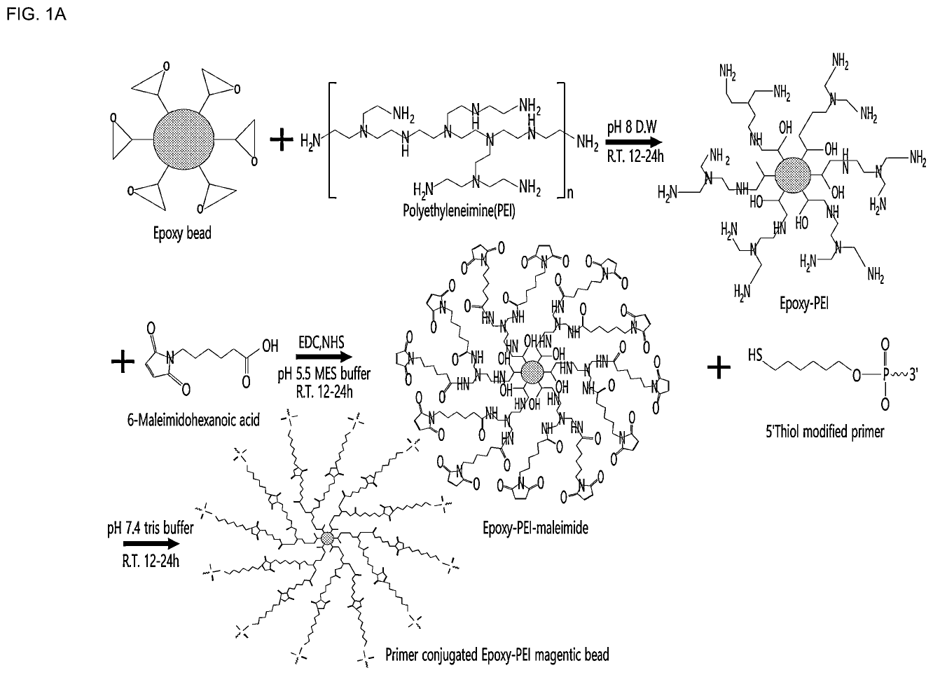

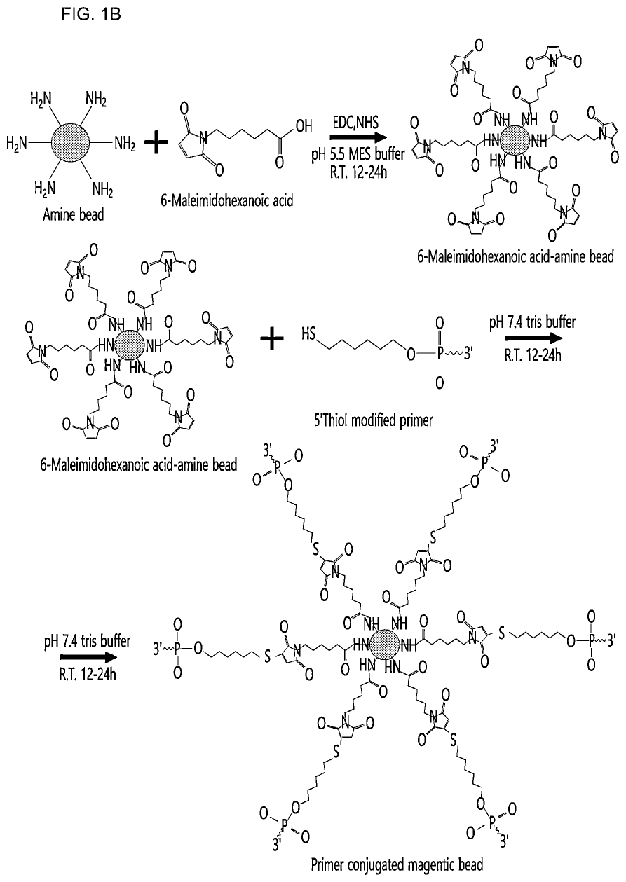

FIG. 1 shows a schematic diagram showing processes of bead synthesis according to an embodiment of the present disclosure.

Specifically, beads (Accunano-Bead™ Epoxy Magnetic nano-beads, size 400 nm, Catalog number (TA-1013-1), Bioneer) having the surface to which an epoxy functional group is connected were dispersed in 3 ml of distilled water at a concentration of 10 mg/ml. Branched polyethyleneimine (PEI) having a molecular weight of 25,000 was dissolved in 2 ml of distilled water at a concentration of 10 mg/ml. In order to bind the epoxy group and the amino group, a solution in which PEI was dissolved was added dropwise to the epoxy beads and reacted at room temperature for 12 hours to 24 hours. The beads (epoxy-PEI beads), in which the reaction was completed, were washed 3 times with a MES buffer at pH 5.5 and stored at a concentration of 10 mg/ml.

1-ethyl-3-(3-dimethylaminopropyl)carbodiimide (EDC), N-hydroxysuccinimide (NHS) were dissolved in 4 ml of pH 5.5 MES buffer in an amount of as much as 100 times the molar equivalent of the primary amino group of PEI, in order to bind the amine group on the bead surface and the carboxyl group of 6-maleimidohexanic acid by using carbodiimide reaction. After that, the solution was added dropwise to the solution in which the beads were dispersed and reacted at room temperature for 12 hours to 24 hours. After the reaction was completed, the beads were washed 3 times with a MES buffer and stored in 100 mM tris buffer at pH 7.4.

In order to bind the maleimide group on the surface of the beads (epoxy-PEI-maleimide beads), in which the reactions were completed, to oligo-nucleotides, oligo-nucleotides substituted with thiol groups at the 5′ end were used. Specifically, for the oligo-nucleotides, primers specific to the HPV L1 site were prepared, and a thiol group was added to each of the 5′ ends. The primers used were purchased from Cosmojintech Korea, and the sequences of the used primers are shown in Table 1 below.

TABLE 1

| Primer name | Nucleotide sequence | SEQ ID NO |
|---|---|---|
| GP6-16 | 5ThioMC6-D/GAAAA | SEQ ID NO: 1 |
| | ATAAA CTGTA AATCA | |
| | TATTC | |
| GP6-50 | 5ThioMC6-D/GCAGT | SEQ ID NO: 2 |
| | TAAGG TTATT TTGCA | |
| | CAGTT GAAAA ATAAA | |
| | CTGTA AATCA TATTC | |

The oligo-nucleotides substituted with a thiol group were reduced for 2 hours in a tris buffer at pH 7.4, in which tris(2-carboxyethyl)phosphine hydrochloride (TCEP) is dissolved at a concentration of 1 μm. 10 mg of the beads with maleimide groups, and 10 nM of the oligo-nucleotides with thiol groups were reacted at room temperature for 12 hours to 24 hours in a pH 7.4 tris buffer. After the reaction was completed, the beads were washed 3 times with a tris buffer and stored in a pH 8.0 tris buffer at a concentration of 10 mg/ml.
Epoxy Bead refers to a bead with an epoxy functional group,
PEI_Epoxy Bead means a bead in which polyethyleneimine is bound to an epoxy bead,
Mal_PEI_Epoxy Bead means a PEI_Epoxy Bead modified with a maleimide group,
GP6-16_Mal_PEI_Epoxy Bead means a bead in which a GP6-16 primer is bound to a Mal_PEI_Epoxy Bead, and
GP6-50_Mal_PEI_Epoxy Bead refers to a bead in which a GP6-50 primer is bound to a Mal_PEI_Epoxy Bead.

### Comparative Example 1. Preparation of NH₂-Maleimide Beads and Oligo-Nucleotide-Bead Complexes

To prepare NH₂-maleimide beads, beads (Accunano-Bead™ NH2 Magnetic nano-beads, size 400 nm, Catalog number (TA-1011-1), Bioneer) having the surface to which an amine functional group is connected were dispersed in a 100 mM MES buffer (pH 5.5) at a concentration of 100 mg/ml. The bead surface was modified by using carbodiimide reaction between the amine group of the bead surface and the carboxyl group of 6-maleimidohexanic acid. Specifically, in order to primarily modify the bead surface with linkers having an amide bond, EDC and NHS were dissolved in 4 ml of MES buffer in an amount of as much as 100 times the molar equivalent of the amino groups connected to the bead surface. After that, the solution was added dropwise to the solution in which the beads were dispersed and reacted at room temperature for 12 hours to 24 hours. After the reaction was completed, the sample was washed three times using a MES buffer and stored in a 100 mM tris buffer, at pH 7.4. Next, oligo-nucleotides were bound to the maleimide groups on the bead surface in the same manner as in Example 1.
NH₂ Bead means a bead with an amine functional group,
Mal_NH₂Bead means an NH₂ Bead modified with a maleimide group,
GP6-16_Mal_NH₂ Bead means a bead in which a GP6-16 primer is bound to a Mal_NH₂ Bead, and
GP6-50_Mal_NH₂ Bead refers to a bead in which a GP6-50 primer is bound to a Mal_NH₂ Bead.

### Comparative Example 2. Silica Bead

As an additional Comparative Example 2, Bioneer's silica beads (Accunano-Bead™ Silica Magnetic nano-beads, size 400 nm, Catalog number (TA-1010-1), Bioneer) were used.

### Experimental Example 1. Morphological Characteristics of Bead Complex

1.1. Determination of Average Particle Size of Bead Complexes

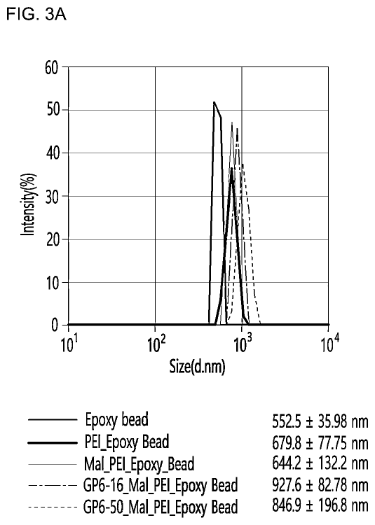

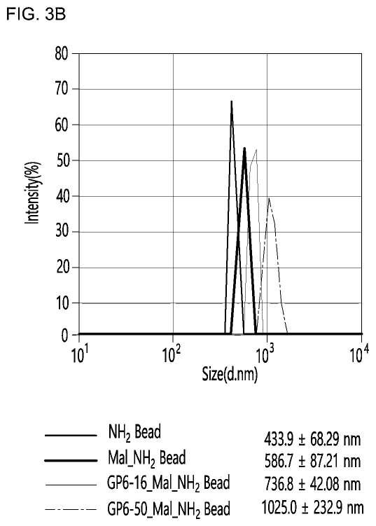

To determine particle sizes of the beads of the bead complexes prepared in Example 1 and Comparative Example 1, average particle sizes were measured by using dynamic light scattering (DLS), and the results are shown in FIGS. 3A and 3B.

Unlike microscopic methods such as scanning electron microscopy (SEM) and transmission electron microscopy (TEM), DLS not only measures the physical diameter, but also measures the thickness including the molecular layer (polymer, surfactant) adsorbed on the particle surface and the solvation layer. Therefore, measurement of the particle size may provide an index that allows indirect determination of whether a change has occurred on the particle surface.

As shown in FIG. 3A, it may be seen that the average particle sizes of Epoxy Beads, PEI_Epoxy Beads, Mal_PEI_Epoxy Beads, GP6-16_Mal_PEI_Epoxy Beads, and GP6-50_Mal_PEI_Epoxy Beads are 552.5, 679.8, 644.2, 927.6, and 846.9, respectively.

As shown in FIG. 3B, it may be seen that the average particle sizes of NH₂ Beads, Mal_NH₂ Beads, GP6-16_Mal_NH₂ Beads, and GP6-50_Mal_NH₂ Beads are 433.9, 586.7, 736.8, and 1025.0, respectively.

As shown in FIGS. 3A and 3B, when a new functional group is conjugated on the surface, a tendency of gradual increase in the size may be confirmed.

1.2. Measuring Average Surface Charge of Beads Using Zetasizer

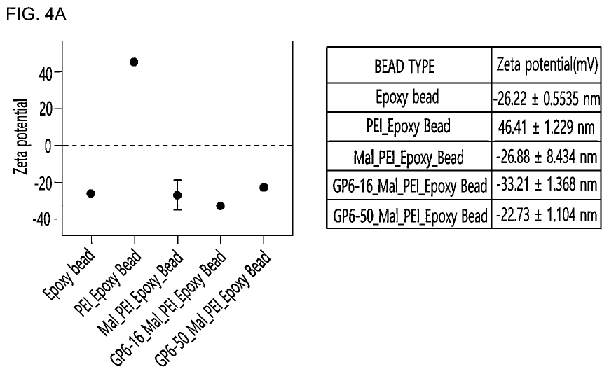

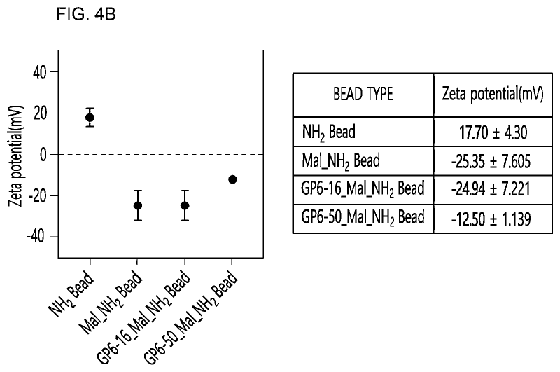

In order to determine an average surface charge of the beads during the manufacturing processes of Example 1 and Comparative Example 1, surface charges were measured by using a Zetasizer, and the results are shown in FIGS. 4A and 4B.

Zeta potential is confirmed by measuring the charge of the liquid layer existing around the particle. There are two liquid layers around the particle, which consists of an inner layer where ions form a strong boundary, and an outer region where ions are weakly bound. The outer region is called a diffuse layer, and the potential of this diffuse layer is called a zeta potential. This zeta potential indicates the degree of repulsive force between charged particles in the dispersion, so it is used as a measure to evaluate stability of the particles. Since surface charge may be measured in addition to stability, the zeta potential is also used as an indicator to confirm whether a new functional group is attached. In the above experiment, it was used as an auxiliary indicator to identify whether a new functional group was attached.

As shown in FIG. 4A, it was found that average surface charges of Epoxy Beads, PEI_Epoxy Beads, Mal_PEI_Epoxy Beads, GP6-16_Mal_PEI_Epoxy Beads, and GP6-50_Mal_PEI_Epoxy Beads were −26.22, 46.41, −26.88, −33.21, and −22.73, respectively.

As shown in FIG. 4B, it may be seen that average surface charges of NH₂ Beads, Mal_NH₂ Beads, GP6-16_Mal_NH₂ Beads, and GP6-50_Mal_NH₂ Beads were 17.70, −25.35, −24.94, and −12.50, respectively.

From the above results, it was confirmed that a new functional group was attached to the bead surface during the manufacturing processes of Example 1 and Comparative Example 1.

### Experimental Example 2. Evaluation of Efficiency for Extracting Nucleic Acid Molecules Using Clinical Specimens

2.1. Detection of Nucleic Acids

In order to confirm capacity to separate DNA of the bead complexes prepared in Example 1 and Comparative Example 1, in which oligo-nucleotides are bound, and the bead complex prepared in Comparative Example 2, collection and recovery efficiency of cell-free DNA (cfDNA) was evaluated.

In order to confirm that the bead complexes prepared in Example 1 and Comparative Example 1 bind specifically to specific cfDNA, a liquid sample having a positive history of human papillomavirus (HPV) and sexually transmitted disease (STD) was prepared. Considering the optimal extraction pH (pH 8 to pH 9) of the bead complex, 1M of pH 8.5 tris buffer, and 200 mM of ethylenediamine-N, N, N′, N′-tetraacetic acid (EDTA) solution were mixed with the liquid sample at a ratio of 1:9. Extraction was performed by using a Kingfisher magmax 96 Cell-Free DNA isolation kit and Kingfisher equipments, according to the Kingfisher magmax protocol.

2.2. Evaluation of Nucleic Acid Extraction Efficiency Using PCR

To confirm whether HPV cfDNA and STD cfDNA were amplified in the extracted cfDNA, Taq polymerases, primers, probes, dNTPs, and Evargreen were added to the extraction solution, and real time PCR (RT-PCR) was performed.

For HPV PCR, 12 μl of a mixture of Invitrogen's platinum II Hot start DNA Taq polymerases, dNTPs, and MgCl₂, 5 μl of primers containing the inventors' company's GP5 & GP6 targeting sequences, 0.8 μl of TE buffer, 1.2 μl of EvaGreen, and 5 μl of urine extract were used.

To perform nucleic acid amplification, each sample was treated at 95° C. for 10 minutes, followed by 45 nucleic acid amplification cycles of 20 seconds at 95° C., 30 seconds at 50° C., and 40 seconds at 72° C., and finally 5 seconds at 60° C. to 95° C.

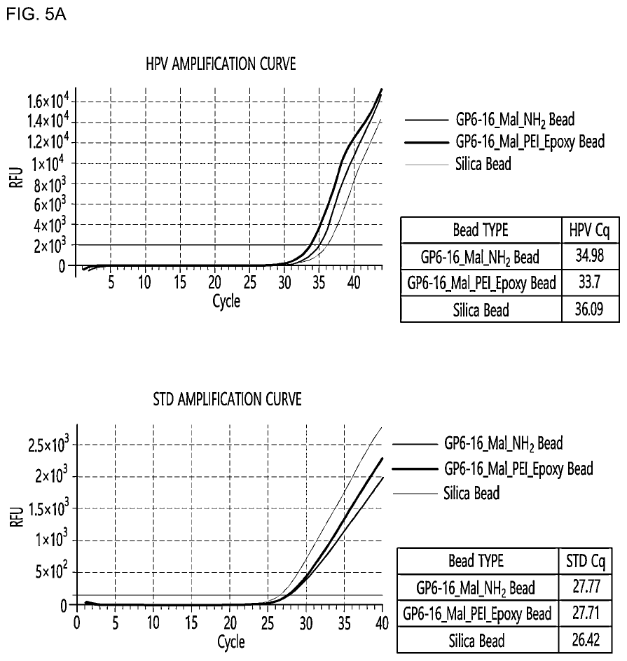

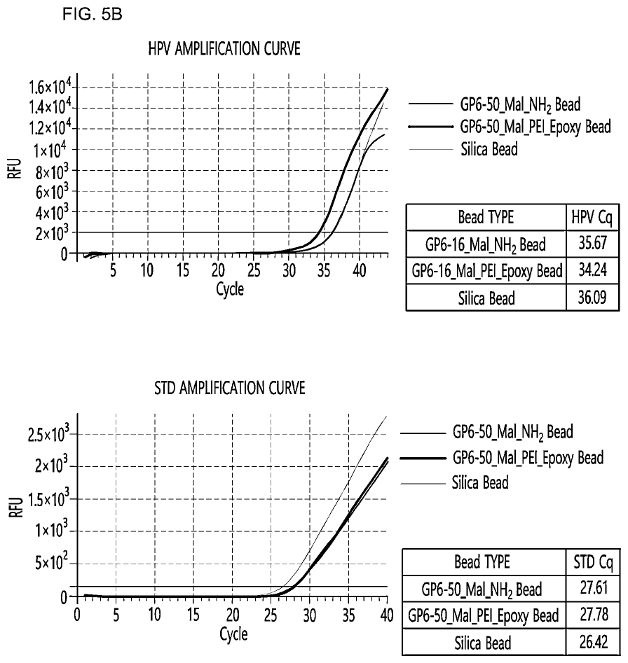

For STD PCR, the inventors' company's Ezplex® STD PCR Kit (in vitro approval No. 18-828 classification number [grade]: N05030.01 [3]) was used. 10 μl of STD RQ mixture, 6 μl of Primer mix, and 4 μl of urine extract were used. In order to perform nucleic acid amplification, each sample was sequentially treated for 2 minutes at 25° C., 2 minutes at 50° C., and 10 minutes at 95° C., followed by 40 cycles of nucleic acid amplification of 20 seconds at 95° C. and 1 minute at 60° C. The results of nucleic acid amplification according to the example are shown in FIG. 5.

As shown in FIG. 5A, the bead complex prepared in Example 1, which is bound to GP6-16, a primer specific to the HPV L1 region, had superior Cq values than Comparative Examples 1 and 2, by 1.28 and 2.39, respectively. In case of STD, it was found that the silica beads of Comparative Example 2 had superior Cq values than Example 1 and Comparative Example 1, by 1.29 and 1.35, respectively.

As shown in FIG. 5B, the bead complex prepared in Example 1 and bound to GP6-50, a primer specific to the HPV L1 region, had superior Cq values than Comparative Examples 1 and 2, by 1.43 and 1.85, respectively. In case of STD, it was found that the silica beads of Comparative Example 2 had superior Cq values than the bead complexes of Example 1 and Comparative Example 1, by 1.36 and 1.19, respectively.

From the above results, it may be seen that the bead complex prepared in Example 1 binds more specifically to HPV cfDNA than the beads of Comparative Examples 1 and 2. These results mean that the bead complex prepared in Example 1 may efficiently extract cfDNA in a sample.

2.3. Evaluation of cfDNA Extraction Efficiency Using Electrophoresis

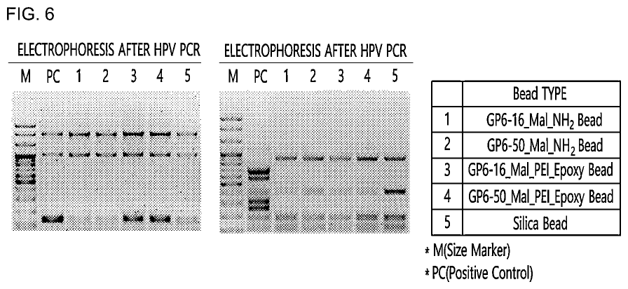

To confirm HPV cfDNA and STD cfDNA from the extracted cfDNA, 4 μl of the extracted solution was loaded and electrophoresed at 150 V for 15 minutes. The results are shown in FIG. 6.

In case of HPV, real-time PCR was performed by using 12 μl of a mixture of Invitrogen's platinum II Hot start DNA Taq polymerases, dNTPs, and MgCl₂, 5 μl of primers with the inventors' company's GP5 & GP6 targeting sequences, 2 μl of TE buffer, and a 5 μl of urine extract. After amplifying the target nucleic acid in the sample by real-time PCR, electrophoresis (150V, 20 minutes) was performed on agarose gel. To perform real-time nucleic acid amplification (polymerase chain reaction; PCR), each sample was sequentially treated at 95° C. for 5 minutes, followed by 45 nucleic acid amplification cycles of 20 seconds at 95° C., 30 seconds at 50° C., and 40 seconds at 72° C., and finally 5 seconds at 60° C. and at 10° C. In case of STD PCR, the inventors' company's Ezplex® STD PCR Kit (in vitro approval No. 18-828 classification number [grade]: N05030.01 [3]) was used. 8 μl of STD RQ mixture, 2 μl of Primer mix, 2 μl of internal control, and 4 μl of urine extract were used. In order to perform nucleic acid amplification, each sample was sequentially treated at 5° C. for 2 minutes and 94° C. for 10 minutes, followed by 40 nucleic acid amplification cycles of 20 seconds at 94° C., 80 seconds at 62° C., and 1 minute at 72° C., and finally 5 seconds at 72° C. and at 4° C. The results are shown in FIG. 6.

As shown in FIG. 6, it was confirmed that when cfDNA was extracted using the bead complex prepared in Example 1, a band corresponding to HPV appeared more clearly in the electrophoresis results, and when cfDNA was extracted using the silica beads of Comparative Example 2, a band corresponding to STD appeared more clearly in the electrophoresis results.

From the above results, it may be seen that the bead complex prepared in Example 1 binds to HPV cfDNA much more specifically than the silica beads of the comparative example. These results mean that the bead complex prepared in Example 1 is capable of efficiently extracting cfDNA in a sample.

### Synthesis Example 1: Synthesis of Bead Complex

Nano-beads (silica beads, Bioneer) having the surfaces to which amine functional groups are connected were dispersed in 1 ml of 100 mM MES buffer (pH 5.5) at a concentration of 100 mg/ml. In order to primarily transform the nano-bead surface with linkers having amide bonds, by using carbodiimide reaction of the amine group at the surface of the nano-bead and the carboxyl group of 6-maleimidohexanic acid, EDC and NHS were dissolved in 4 ml of MES buffer in an amount of as much as 100 times the molar equivalent of the amino groups connected to the surface of the nano-bead. Afterwards, the solution was added dropwise to the solution in which the nano-beads were dispersed and reacted at room temperature for 12 hours to 24 hours. After the reaction was completed, the sample was washed 3 times using a MES buffer and stored in 100 mM of tris buffer at pH 7.4. In order to connect primers with the maleimide groups on the surface of the intermediate nano-bead, in which the primary linker modification reaction was completed, the primers (sequence specific to human papillomavirus (HPV) L1 region, purchased from Cosmogintech Korea), in which the 5′ end is modified with a 1-hexyl thiol group, were used. The primers, in which the 5′ end was modified with a 1-hexyl thiol group (5′-TTTNTNACNKKNGTNGAYACNAC-3′, SEQ ID NO: 3, N is ideoxyl thyimine, GP5+ primer or thiol GP5+ primer), were reduced for 2 hours in a pH 7.4 buffer, in which tris (2-carboxylethyl)phosphine hydrochloride (TCEP) at a concentration of 1 μm was dissolved. 10 mg of the nano-beads primarily modified to have maleimide groups and 10 nM of the primers with thiol groups were reacted at room temperature in a pH 7.4 tris buffer for 12 hours to 24 hours. After the reaction was completed, the nano-beads were washed 3 times using the tris buffer, and stored in a pH 8.0 tris buffer at a concentration of 10 mg/ml. Hereinafter, the bead complex prepared in Synthesis Example 1 is referred to as Thiol GP5+_NH₂ bead.

### Synthesis Example 2: Synthesis of Bead Complex

Except for using a primer with the 5′ end modified with a 1-hexyl thiol group (5′-GAAANAYNAANTGYANNWCRWAYTCYTC-3′, SEQ ID NO: 4, hereinafter, GP6+ primer or Thiol GP6+ primer), in place of the GP5+ primer, a bead complex was prepared by repeating the procedures of Synthesis Example 1. Hereinafter, the bead complex prepared in Synthesis Example 2 is referred to as Thiol GP6+_NH₂ bead.

### Comparative Synthesis Example 1: Amino Group-Bonded Nano-Beads

Nano-beads having the surfaces to which amine groups are connected were used as they were without surface modification and connection with a primer. Hereinafter, the nano-beads of Comparative Synthesis Example 1 are referred to as NH₂ beads.

### Comparative Synthesis Example 2: Amide-Bonded Nano-Beads

Nano-beads, in which the surface is modified with linkers having an amide bond using carboiimide reaction of the amino groups connected to the surface of the nano-bead and carboxyalic acid of 6-maleimidohxexanoic acid, were used. Hereinafter, the nano-beads prepared in Comparative Synthesis Example 2 are referred to as NH2-Maleimide beads.

### Comparative Synthesis Example 3: Hydroxy Group Silica Beads

Nano-beads (silica beads, bioneers) having hydroxyl groups on the surface were used as they were without surface modification and connection with a primer. Hereinafter, the beads prepared in Comparative Synthesis Example 3 are referred to as COOH beads.

### Comparative Synthesis Example 4: Synthesis of Amide Bead Complex

Nano-beads (COOH magnetic beads, Bioneer) having carboxylic acid present on the surface and oligo-nucleotides, in which the 3′ end is substituted with an amine group, were bonded using carbodiimide reaction. The nano-beads (silica beads, Bioneer) connected to carboxylic acid on the surface were dispersed in 1 ml of 100 mM MES buffer (pH 5.5) at a concentration of 100 mg/ml. In order to connect primers with the carboxyl group on the surface of the nano-bead, primers (sequence specific to human papillomavirus (HPV) L1 region, GP5+ primer, purchased from Cosmogintech Korea), with the 5′ end modified with an amine group, were used. The procedures of Synthesis Example 1 were repeated to prepare a bead complex. The bead complex prepared in Comparative Synthesis Example 4 is referred to as an NH₂ GP5+_COOH bead.

### Comparative Synthesis Example 5: Preparation of Bead Complexes

The procedures of Synthesis Example 1 were repeated, except that GP6+ primers, in which the 5′ end is modified with an amino group, were used in place of the GP5+ primers, to prepare bead complexes. Hereinafter, the bead complex prepared in Comparative Synthesis Example 5 is referred to as an NH2 GP6+_COOH bead.

### Comparative Synthesis Example 6: Silica Beads

Nano-beads (silica beads, Bioneer) that were not modified with a functional group on the surface were used as they were without surface modification and connection with a primer. The beads prepared in Comparative Synthesis Example 6 are referred to as silica beads.

### Experimental Example 3: Measurement of Average Particle Sizes of Bead Complexes

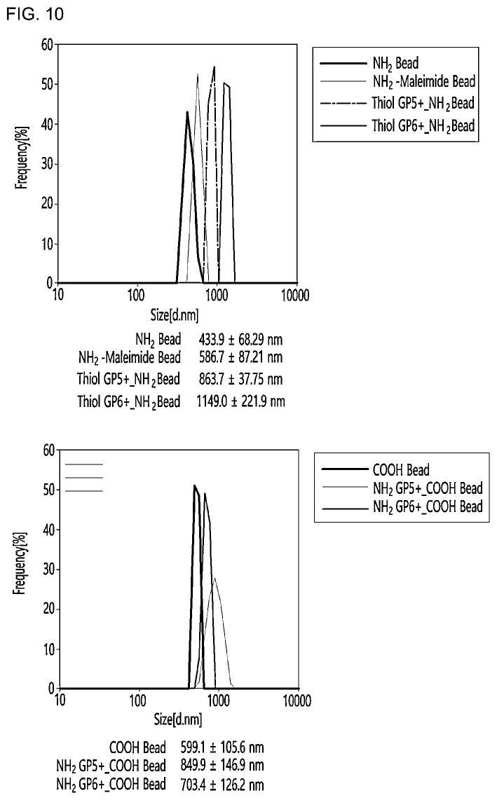

Average particle sizes of each of the bead complexes finally prepared in Synthesis Example 1 and Synthesis Example 2, and each of the bead complexes prepared in Comparative Synthesis Examples 1 to 5, were measured by using a dynamic light scattering (DLS) method. The results of measuring average particle sizes of the bead complexes and silica beads are shown in FIG. 10. As shown in FIG. 10, it was confirmed that sizes of the bead complexes according to Synthesis Example 1 and Synthesis Example 2, to which a primer was conjugated by an amide bond and an sulfide bond on the surface of the nano-beads, were greatly increased.

### Experimental Example 4: Measurement of Average Surface Charges of Bead Complexes

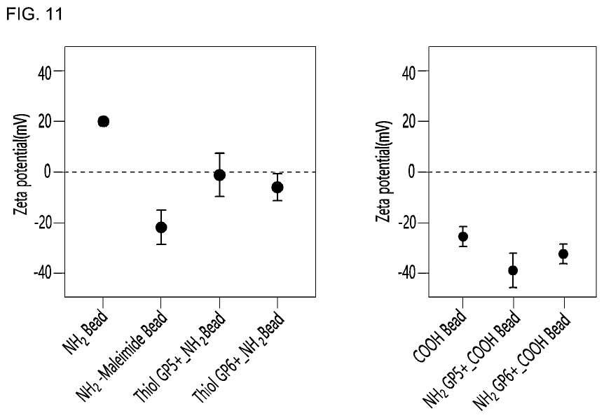

Average surface charges of each of the bead complexes finally prepared in Synthesis Example 1 and Synthesis Example 2, and each of the bead complexes prepared in Comparative Synthesis Examples 1 to 5, were measured by using a Zetasizer. The measurement results are shown in FIG. 11. From the measurement results, it was confirmed that the average surface charge was changed as the linkers on the surface of the nano-bead were changed.

### Experimental Example 5: Evaluation of Extraction Efficiency for Target Nucleic Acid Molecules Using Clinical Specimens

In order to confirm capacities to separate nucleic acids of each of the bead complexes prepared in Synthesis Example 1 and Synthesis Example 2, and each of the bead complexes prepared in Comparative Synthesis Examples 3 to 6, collection and recovery efficiency of cell-free DNA (cfDNA) was evaluated. In order to confirm that the bead complexes prepared in Synthesis Example 1 and Synthesis Example 2 bind specifically to a specific cfDNA, a cervix swab sample with a positive history of HPV and STD was prepared. The extraction experiment was carried out by diluting the prepared sample in negative urine. Considering the optimal pH (pH 8 to pH 9) for extracting cfDNA of the bead complex, 1 M of pH 8.5 tris buffer, and 200 mM of EDTA solution was mixed with urine in a 1:9 ratio. Extraction was performed by using a Kingfisher magmax 96 Cell-Free DNA isolation kit and Kingfisher equipments, according to the Kingfisher magmax protocol.

(1) Evaluation of cfDNA Extraction Efficiency Using PCR

To confirm whether HPV cfDNA and STD cfDNA were amplified in the above-extracted cfDNA, Taq polymerases, primers, probes, dNTPs, and Evargreen were added to the extraction solution, and real time PCR (RT-PCR) was performed. For HPV PCR, 12 μl of a mixture of Invitrogen's platinum II Hot start DNA Taq polymerases, dNTPs, and MgCl₂, 5 μl of primers containing the inventors' company's GP5 & GP6 targeting sequences, 0.8 μl of TE buffer, 1.2 μl of EvaGreen, and 5 μl of urine extract were used. To perform nucleic acid amplification, each sample was treated at 95° C. for 10 minutes, followed by 45 nucleic acid amplification cycles of 20 seconds at 95° C., 30 seconds at 50° C., and 40 seconds at 72° C., and finally 5 seconds at 60° C. to 95° C.

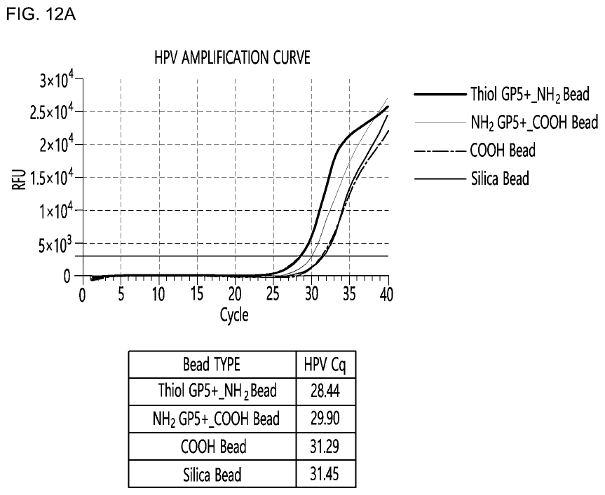

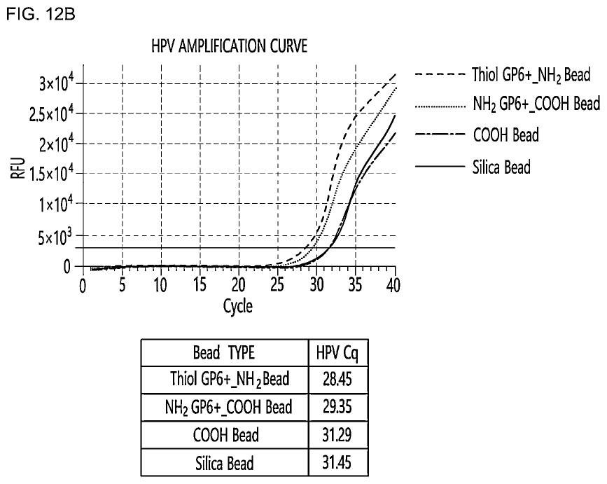

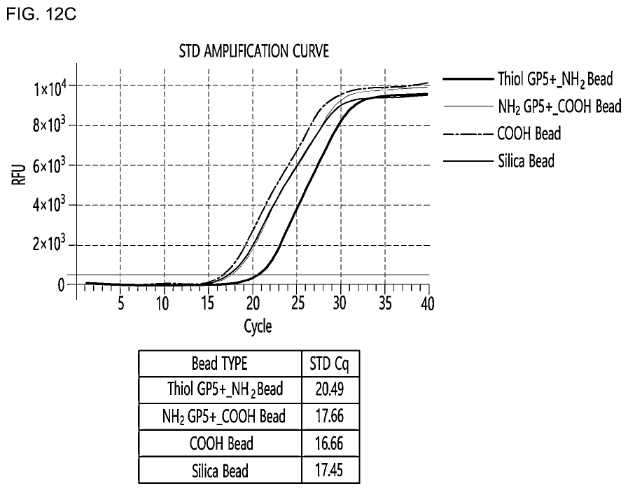

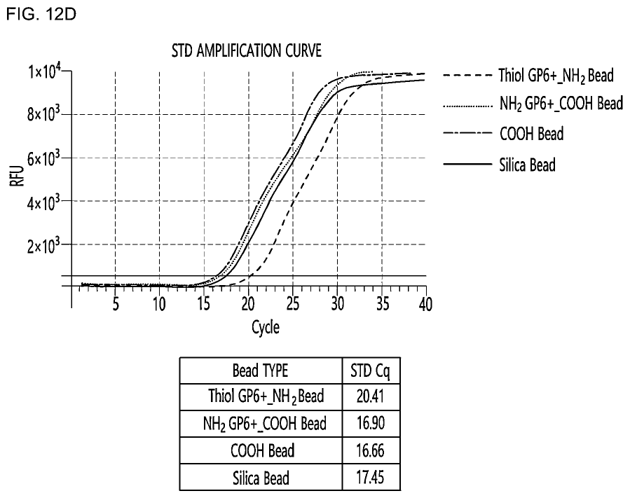

In case of STD PCR, the inventors' company's Ezplex® STD PCR Kit (in vitro approval No. 18-828 classification number [grade]: N05030.01 [3]) was used. 10 μl of STD RQ mixture, 6 μl of Primer mix, and 4 μl of urine extract were used. In order to perform nucleic acid amplification, each sample was sequentially treated for 2 minutes at 25° C., 2 minutes at 50° C., and 10 minutes at 95° C., followed by 40 cycles of nucleic acid amplification of 20 seconds at 95° C. and 1 minute at 60° C. The results of nucleic acid amplification according to the example are shown in FIG. 12.

As shown in FIG. 12, the bead complexes to which primers specific to the HPV L1 region were bound according to Synthesis Examples 1 and 2 were confirmed to have superior Cq values compared to the COOH bead of Comparative Synthesis Example 3 which was not bound to a specific primer for HPV cfDNA or the silica beads of Comparative Synthesis Example 6. Conversely, in the case of STD cfDNA, it was confirmed that the COOH beads of Comparative Synthesis Example 1 or the silica beads of Comparative Synthesis Example 6 had superior Cq values than the bead complexes of Synthesis Examples 1 and 2. On the other hand, the bead complexes synthesized in Synthesis Examples 1 and 2 amplified the intended HPV nucleic acid more rapidly and efficiently, compared to the bead complexes in which an amide bond was formed between an amino group and a carboxylic acid group without a linker in Comparative Synthesis Examples 4 to 5. From these results, it was confirmed that the bead complexes of Synthesis Examples 1 and 2 were more specifically bound to HPV cfDNA than the beads or bead complexes of Comparative Synthesis Examples.

(2) Evaluation of cfDNA Extraction Efficiency by Using Electrophoresis

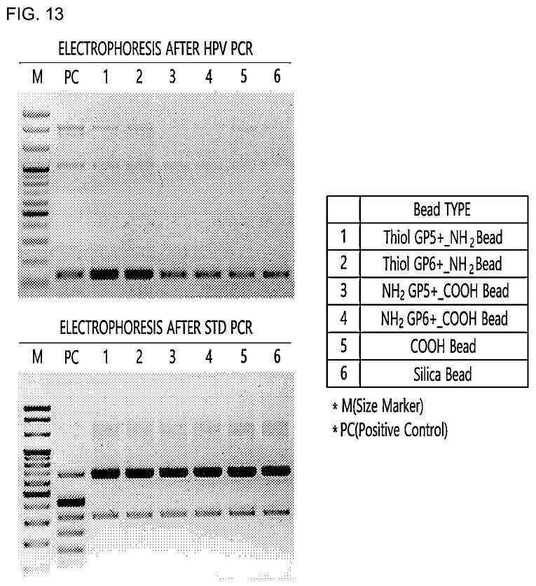

In order to confirm HPV cfDNA and STD cfDNA from the cfDNA extracted above, 4 μl of the extracted solution was loaded and electrophoresed at 150V for 20 minutes. The evaluation results of the electrophoresis are shown in FIG. 13.

For HPV, real-time PCR was performed using 12 μl of a mixture, in which Invitrogen's platinum II Hot start DNA Taq polymerases, dNTPs, and MgCl₂ are mixed, 5 μl of primers containing the inventors' company's GP5 & GP6 targeting sequence, 2 μl of TE buffer, and 5 μl of urine extract. After amplifying the target nucleic acid in the sample by real-time PCR, electrophoresis (150V, 20 minutes) was performed on agarose gel. To perform real-time nucleic acid amplification (polymerase chain reaction; PCR), each sample was sequentially treated at 95° C. for 5 minutes, followed by 45 nucleic acid amplification cycles of 20 seconds at 95° C., 30 seconds at 50° C., and 40 seconds at 72° C., and finally 5 seconds at 60° C. and at 10° C.

In the case of STD PCR, the inventors' company's Ezplex® STD PCR Kit (in vitro approval No. 18-828 classification number [grade]: N05030.01 [3]) was used. 8 μl of STD RQ mixture, 2 μl of Primer mix, 2 μl of Internal control, and 4 μl of urine extract were used. In order to perform nucleic acid amplification, each sample was sequentially treated at 5° C. for 2 minutes and 94° C. for 10 minutes, followed by 40 nucleic acid amplification cycles of 20 seconds at 94° C., 80 seconds at 62° C., and 1 minute at 72° C., and finally 5 seconds at 72° C. and at 4° C.

As shown in FIG. 13, with bead complexes synthesized in Synthesis Example 1 and Synthesis Example 2, HPV bands were more clearly shown, and an STD band was more clearly shown by the silica beads of Comparative Synthesis Example 6. From these results, it was confirmed that each of the bead complexes prepared in Synthesis Example 1 and Synthesis Example 2 bind to HPV cfDNA more specifically than the beads or bead complexes prepared in Comparative Synthesis Examples.

In the above, the present disclosure has been described based on example embodiments and examples of the present disclosure, but the present disclosure is not limited to the technical ideas described in the above embodiments and examples. Rather, those of ordinary skill in the art to which the present disclosure pertains may easily deduce various modifications and changes based on the above-described embodiments and examples. However, it is clear from the appended claims that all such modifications and changes are within the scope of the present disclosure.

1. A bead complex in which one end of an oligo-nucleotide, which binds specifically to a target nucleic acid molecule, is conjugated to a surface of a bead, and the surface of the bead and the oligo-nucleotide is bonded by a structure of Formula 1 below: 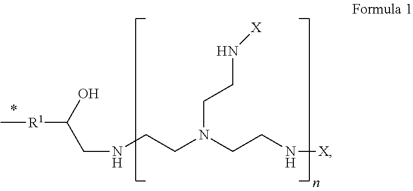
wherein, X in the formula is hydrogen or 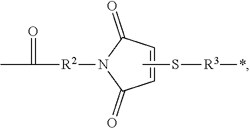
and at least one X is 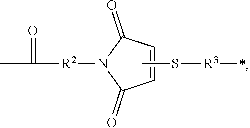
R¹ in the formula is a direct bond or a C₁-C₂₀ aliphatic hydrocarbon group,
R² and R³ in the formula are each independently a C₂-C₂₀ aliphatic hydrocarbon group,
n in the formula is an integer of 1 or more, for example, an integer of 1 to 100,000, 1 to 10,000, 1 to 1,000, 1 to 100, 1 to 50, 1 to 40, 1 to 30, 1 to 20, or 1 to 10, and
the asterisk on the left of R¹ indicates a site connected to the bead surface, and the asterisk on the right of R³ indicates a site connected to the end of the oligo-nucleotide.

2. The bead complex of claim 1, wherein R² is a C₂-C₁₀ alkylene group.

3. The bead complex of claim 1, wherein the bead consists of inorganic or organic materials.

4. The bead complex of claim 1, wherein the bead is a magnetized bead.

5. The bead complex of claim 1, wherein the target nucleic acid molecule is derived from a cancer cell or a pathogen.

6. The bead complex of claim 5, wherein the pathogen comprises pathogenic viruses and pathogenic bacteria.

7. The bead complex of claim 1, wherein the target nucleic acid molecule is a nucleic acid molecule as a marker indicating an onset of cancer or an infection of a pathogen-related disease.

8. The bead complex of claim 1, wherein the target nucleic acid molecule is cell-free nucleic acid.

9. The bead complex of claim 1, wherein the oligo-nucleotide is modified with a thiol group at the 5′ end or the 3′ end.

10. The bead complex of claim 1, wherein the oligo-nucleotide consists of 20 to 100 nucleotides.

11. A use of the bead complex of claim 1 for detection of a target nucleic acid molecule.

12. A method of preparing the bead complex of claim 1, comprising:
(a) transforming the surface of the bead into a structure of Formula 3 which is modified with an amino group, by reacting polyethyleneimine (PEI) with the bead having a surface to which an epoxy group represented by Formula 2 is connected;
(b) transforming the surface of the bead into a structure of Formula 5 which is modified with a maleimide group, by reacting carboxylic acid of Formula 4 with the bead having a structure of Formula 3, which is modified with an amino group; and
(c) conjugating by reacting a bead having the structure of Formula 5 which is modified with a maleimide group, with an oligo-nucleotide that binds specifically to a target nucleic acid molecule and has an end modified with aliphatic thiol of Formula 6: 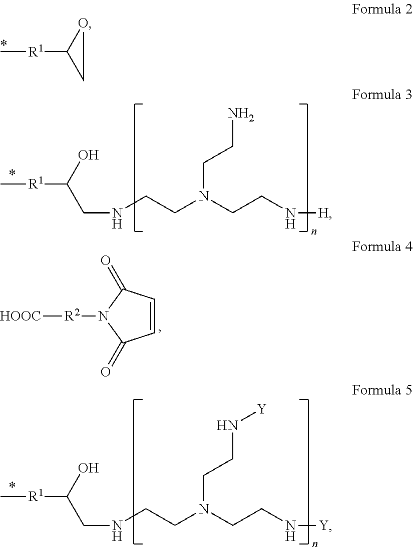
wherein Y in Formula 5 is hydrogen or 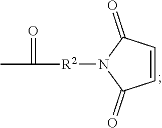
and at least one Y is 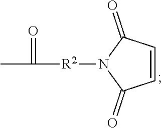
HS-R³—* Formula 6
R¹ in Formulas 2 to 6 is a direct bond or a C₁-C₂₀ aliphatic hydrocarbon group,
R² and R³ in the formula are each independently a C₂-C₂₀ aliphatic hydrocarbon group,
n in the formula is an integer from 1 to 100,000,
the asterisk on the left of R¹ indicates a site connected to the bead surface, and the asterisk on the right of R³ indicates a site connected to the end of the oligo-nucleotide.

13. A kit for detecting a target nucleic acid molecule in a biological sample comprising the bead complex of claim 1.

14. A method of detecting a target nucleic acid molecule in a biological sample, comprising: reacting the bead complex of claim 1 with a biological sample;
separating the bead complex, in which the target nucleic acid present in the biological sample and the oligo-nucleotide are bound; and
detecting whether the oligo-nucleotide and the target nucleic acid molecule are bound.

15. The method of claim 14, wherein the biological sample comprises urine, saliva, sputum, blood and nasopharyngeal smear.

16. The method of claim 14, wherein the process of detecting whether the oligo-nucleotide and the target nucleic acid molecule are bound comprises, performing polymerase chain reaction (PCR) using the target nucleic acid molecule bound to the oligo-nucleotide as a template.

17. A bead complex, in which one end of an oligo-nucleotide binding specifically to a target nucleic acid molecule is conjugated to a surface of a nano-bead, and a linker having a structure of Formula 7 below is interposed between the surface of the nano-bead and the oligo-nucleotide: 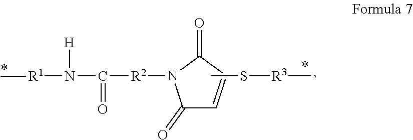
wherein, in Formula 7, R¹ is a direct bond or a C₁-C₂₀ aliphatic hydrocarbon group; R² and R³ are each independently a C₃-C₂₀ divalent aliphatic hydrocarbon linker; the asterisk on the left of R¹ indicates a site connected to the surface of the nano-bead, and the asterisk on the right of R³ indicates the site connected to the end of the oligo-nucleotide.

18. A method of preparing the bead complex of claim 17, comprising:
transforming the surface of the nano-bead with a linker having a structure of Formula 10 by reacting the nano-bead having a surface to which an amino group represented by Formula 8 is connected, with a carboxylic acid represented by Formula 9; and
reacting the nano-bead, of which surface is modified with the linker having the structure of Formula 10, with an oligo-nucleotide that binds specifically to a target nucleic acid molecule and has an end modified with aliphatic thiol of Formula 11. 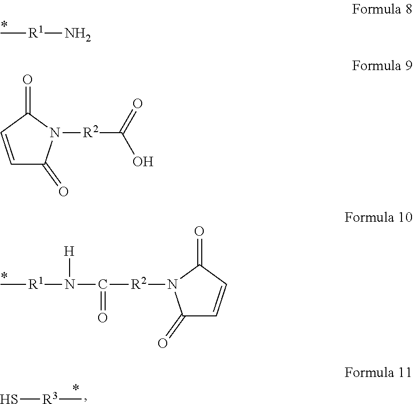
wherein R¹, R², R³, and the asterisk in Formulas 8 to 11 are each the same as defined in claim 17.
